SEQUENCE LISTING (9 sequences)

SEQ ID NO: 1 - DNA, 2460 bp, Escherichia coli
atgtattcaagtagcagaaaaaggtgcccgaaaaccaaatgggctttgaaacttcttactgccgcatttttagcagcgagtcccgcggcgaagagtgctgttaataacgcctatgatgcattgattattgaagctcgcaagggtaatactcagccagctttgtcatggtttgcactaaaatcagcactcagcaataaccaaattgctgactggttacagattgccttatgggccgggcaagataaacaggttattaccgtttacaaccgctaccgtcatcagcaattaccagcgcgtggttatgcagctgtcgccgtcgcttatcgtaacctgcaacaatggcaaaactcgcttacactgtggcaaaaggcgctctctctggagccgcaaaataaggattatcaacggggacaaattttaaccctggcagatgctggtcactatgatactgcgctggttaaacttaagcagcttaactctggagcaccggacaaagccaatttactcgcagaagcctatatctataaactggcggggcgtcatcaggatgaattacgggcgatgacagagtcattacctgaaaatgcatctacgcaacaatatcccacagaatacgtgcaggcattacgtaataatcaacttgctgccgcgattgacgatgccaatttaacgccagatattcgcgctgatattcatgccgaactggtcagactgtcgtttatgcctacgcgcagtgaaagtgaacgttatgccattgccgatcgcgccctcgcccaatacgctgcattagaaattctgtggcacgataacccagaccgcactgcccagtaccagcgtattcaggttgatcatcttggcgcgttattaactcgcgatcgttataaagacgttatttctcactatcagcgattaaaaaagacggggcaaattattccgccctgggggcaatattgggttgcatcggcttatctcaaagatcatcagccgaaaaaagcacagtcaataatgaccgagctcttttatcacaaggagaccattgccccggatttatccgatgaagaacttgcggatctcttttacagccacctggagagtgaaaattatccgggcgcgctaactgtcacccaacataccattaatacttcgccgcctttccttcggttaatgggcacgcctacgagcatcccgaatgatacctggttacaggggcattcgtttctctcaaccgtagcaaaatatagtaatgatcttcctcaggctgaaatgacagccagagagcttgcttataacgcaccaggaaatcagggactgcgcattgattacgcgagtgtgttacaagcccgcggttggcctcgtgcagcagaaaatgaattaaaaaaagcagaagtgatcgagccacgtaatattaatctggaggttgaacaagcctggacagcattaacgttacaagaatggcagcaggcagctgtcttaacgcacgatgttgtcgaacgtgaaccgcaagatcccggcgttgtacgattaaaacgtgcggttgatgtacataatcttgcagagcttcgtatcgctggctcaacaggaattgatgccgaaggcccggatagtggtaaacatgatgtcgacttaaccaccatcgtttattcaccaccgctgaaggataactggcgcggttttgctggattcggttatgccgatggacaatttagcgaaggaaaagggattgttcgcgactggcttgcgggtgttgagtggcggtcacgtaatatctggctcgaggcagagtacgctgaacgcgttttcaatcatgagcataaacccggcgcgcgcctgtctggctggtatgattttaatgataactggcgtattggttcgcaactggaacgcctctctcaccgcgttccattacgggcaatgaaaaatggtgttacaggcaacagtgctcaggcttatgttcgctggtatcaaaatgagcggcgtaagtacggtgtctcctgggctttcactgatttttccgacagtaaccagcgtcatgaagtctcacttgagggtcaggaacgcatctggtcttcaccatatttgattgtcgatttcctacccagtctgtattacgaacaaaatacagaacacgataccccatactacaaccctataaaaacgttcgatattgttccggcatttgaggcaagccatttgttatggcgaagctatgaaaatagctgggagcaaatattcagcgcaggtgttggtgcctcctggcaaaaacattatggcacggatgtcgtcacccaactcggctacgggcaacgcattagttggaatgacgtgattgatgctggcgcaacgctacgctgggaaaaacgaccttatgacggtgacagagaacacaacttatacgttgaattcgatatgacattcagattttaaggataaatatgttacgtaatggaaataaatatctcc

SEQ ID NO: 2 - protein, 807 aa, Escherichia coli
MYSSSRKRCPKTKWALKLLTAAFLAASPAAKSAVNNAYDALIIEARKGNTQPALSWFALKSALSNNQIADWLQIALWAGQDKQVITVYNRYRHQQLPARGYAAVAVAYRNLQQWQNSLTLWQKALSLEPQNKDYQRGQILTLADAGHYDTALVKLKQLNSGAPDKANLLAEAYIYKLAGRHQDELRAMTESLPENASTQQYPTEYVQALRNNQLAAAIDDANLTPDIRADIHAELVRLSFMPTRSESERYAIADRALAQYAALEILWHDNPDRTAQYQRIQVDHLGALLTRDRYKDVISHYQRLKKTGQIIPPWGQYWVASAYLKDHQPKKAQSIMTELFYHKETIAPDLSDEELADLFYSHLESENYPGALTVTQHTINTSPPFLRLMGTPTSIPNDTWLQGHSFLSTVAKYSNDLPQAEMTARELAYNAPGNQGLRIDYASVLQARGWPRAAENELKKAEVIEPRNINLEVEQAWTALTLQEWQQAAVLTHDVVEREPQDPGVVRLKRAVDVHNLAELRIAGSTGIDAEGPDSGKHDVDLTTIVYSPPLKDNWRGFAGFGYADGQFSEGKGIVRDWLAGVEWRSRNIWLEAEYAERVFNHEHKPGARLSGWYDFNDNWRIGSQLERLSHRVPLRAMKNGVTGNSAQAYVRWYQNERRKYGVSWAFTDFSDSNQRHEVSLEGQERIWSSPYLIVDFLPSLYYEQNTEHDTPYYNPIKTFDIVPAFEASHLLWRSYENSWEQIFSAGVGASWQKHYGTDVVTQLGYGQRISWNDVIDAGATLRWEKRPYDGDREHNLYVEFDMTFRF

SEQ ID NO: 3 - DNA, 2031 bp, Escherichia coli
ttaaggataaatatgttacgtaatggaaataaatatctcctgatgctggtgagtataattatgctcaccgcgtgcattagccagtcaagaacatcatttataccgccacaggatcgcgaatctttactcgccgagcaaccgtggccgcataatggttttgtagcgatttcatggcataacgttgaagacgaagctgccgaccagcgttttatgtcagtgcggacatcagcactgcgtgaacaatttgcctggctgcgcgagaacggttatcaaccggtcagtattgctcaaattcgtgaagcacatcgaggaggaaaaccgctaccggaaaaagctgtagtgctgacttttgatgacggctaccagagtttttatacccgcgtcttcccaattcttcaggccttccagtggcctgctgtatgggcccccgtcggcagttgggtcgatacgccagcggataaacaagtaaaatttggcgatgagttggtcgatcgagaatattttgccacgtggcaacaagtgcgagaagttgcgcgttcccggctcgttgagctcgcttctcatacatggaattctcactacggtattcaggctaatgccaccggcagcttattgcctgtatatgtaaatcgtgcatattttactgaccacgcacggtatgaaaccgcagcagaataccgggaaagaattcgtctggatgctgtaaaaatgacggaatacctgcgtacaaaggttgaggtaaatccacacgtttttgtttggccttatggcgaagcgaatggcatagcgatagaggaattaaaaaaactcggttatgacatgttcttcacccttgaatcaggtttggcaaatgcgtcgcaattggattccattccgcgggtattaatcgccaataatccctcattaaaagagtttgcccagcaaattattaccgtacaggaaaaatcaccacaacggataatgcatatcgatcttgattacgtttatgacgaaaacctccagcaaatggatcgcaatattgatgtgctaattcagcgggtgaaagatatgcaaatatcaaccgtgtatttgcaggcatttgctgatcccgatggtgatgggctggtcaaagaggtctggtttccaaatcgtttgctaccaatgaaagcagatatttttagtcgggttgcctggcaattacgtacccgctcaggtgtaaacatctatgcgtggatgccggtattaagctgggatttagatcccacattaacgcgagtaaaatacttaccaacaggggagaaaaaagcacaaattcatcctgaacaatatcaccgtctctctcctttcgatgacagagtcagagcacaagttggcatgttatatgaagatcttgccggacatgctgcttttgatggcatattgttccacgatgatgctttgctttcagattatgaagatgccagtgcaccggctatcacggcttatcagcaagcaggctttagcgggagtctgagcgaaattcgacaaaacccggagcaatttaaacagtgggcccgctttaaaagtcgtgcgttaactgacttcactttagaacttagtgcgcgcgtaaaagccattcgcggtccacatattaaaactgcacgaaatatttttgcacttccggtaatacaacctgaaagtgaagcctggtttgcacagaattatgctgatttcctaaaaagctatgactggaccgctattatggctatgccttatctggaaggtgtcgcagaaaaatcggctgaccaatggttaatacaattgaccaatcaaattaaaaacatccctcaggctaaagacaaatctattttagaattacaggcacaaaactggcagaaaaatggtcagcatcaggctatttcttcgcaacaactcgctcactggatgagcctattacaactgaatggagtgaaaaactatggttattatcccgacaattttctgcataaccaacctgaaatagaccttattcgtcctgagttttcaacagcctggtatccgaaaaatgattaa

SEQ ID NO: 4 - protein, 672 aa, Escherichia coli
MLRNGNKYLLMLVSIIMLTACISQSRTSFIPPQDRESLLAEQPWPHNGFVAISWHNVEDEAADQRFMSVRTSALREQFAWLRENGYQPVSIAQIREAHRGGKPLPEKAVVLTFDDGYQSFYTRVFPILQAFQWPAVWAPVGSWVDTPADKQVKFGDELVDREYFATWQQVREVARSRLVELASHTWNSHYGIQANATGSLLPVYVNRAYFTDHARYETAAEYRERIRLDAVKMTEYLRTKVEVNPHVFVWPYGEANGIAIEELKKLGYDMFFTLESGLANASQLDSIPRVLIANNPSLKEFAQQIITVQEKSPQRIMHIDLDYVYDENLQQMDRNIDVLIQRVKDMQISTVYLQAFADPDGDGLVKEVWFPNRLLPMKADIFSRVAWQLRTRSGVNIYAWMPVLSWDLDPTLTRVKYLPTGEKKAQIHPEQYHRLSPFDDRVRAQVGMLYEDLAGHAAFDGILFHDDALLSDYEDASAPAITAYQQAGFSGSLSEIRQNPEQFKQWARFKSRALTDFTLELSARVKAIRGPHIKTARNIFALPVIQPESEAWFAQNYADFLKSYDWTAIMAMPYLEGVAEKSADQWLIQLTNQIKNIPQAKDKSILELQAQNWQKNGQHQAISSQQLAHWMSLLQLNGVKNYGYYPDNFLHNQPEIDLIRPEFSTAWYPKND

SEQ ID NO: 5 - DNA, 1380 bp, Escherichia coli
aaaatgattaatcgcatcgtatcgttttttatattatgtctggtgttatgcatacccctatgcgtagcgtactttcactctggtgaactgatgatgaggttcgttttcttctggccgttttttatgtccattatgtggattgttggcggcgtctatttctgggtctatcgtgaacgccactggccgtggggagaaaacgcaccagctccccagttgaaagataatccgtctatctccattatcattccctgttttaatgaggagaaaaacgttgaggaaaccatacacgccgctttagcacagcgttatgagaacattgaagttattgccgtaaatgacggttcaacagataaaacccgtgccatcctggatcgcatggctgcacaaattccccatttgcgggtcattcatctggcgcaaaaccaggggaaagccattgcgcttaaaaccggagctgccgcggcgaaaagtgaatatctggtgtgcattgatggcgatgcgttattagaccgcgatgcggcggcatatattgtggaaccgatgttgtacaacccgcgtgtgggtgccgtaaccggtaatcctcgtattcgaacacgttctaccctggtgggtaaaattcaggttggcgagtattcctcaattattggtttgatcaagcgaacccagcgtatctatggaaacgtatttaccgtttccggtgttattgccgcatttcgtcgcagcgccctggcagaagtgggttactggagtgacgatatgatcaccgaagatattgatattagctggaagctgcagttgaatcagtggacgattttttacgagccacgggcactgtgctggatattaatgcctgaaacgttaaaagggctgtggaaacagcgcctgcgctgggctcagggcggtgcagaagtattcctcaaaaatatgacaaggttgtggcgcaaagaaaactttcgaatgtggccgctgttttttgaatactgcctgacgacaatatgggccttcacctgcctggtcggtttcattatttacgcagtccaacttgccggtgtaccgttaaatattgaattgacacatatcgctgcgacacatactgccggaatattattgtgtacgttatgtttactgcaatttattgtcagcctgatgatcgagaatcgctatgagcataatctgacttcatcgcttttctggattatttggttcccggttattttctggatgctgagcctggcaacgacattggtatcatttacacgagtcatgttgatgcctaaaaagcaacgcgcccgttgggtaagtcccgatcgcgggattctgagaggttaatatgaacaatttaattattacgacccgacaatcaccagtacgtttactggt

SEQ ID NO: 6 - protein, 445 aa, Escherichia coli
MINRIVSFFILCLVLCIPLCVAYFHSGELMMRFVFFWPFFMSIMWIVGGVYFWVYRERHWPWGENAPAPQLKDNPSISIIIPCFNEEKNVEETIHAALAQRYENIEVIAVNDGSTDKTRAILDRMAAQIPHLRVIHLAQNQGKAIALKTGAAAAKSEYLVCIDGDALLDRDAAAYIVEPMLYNPRVGAVTGNPRIRTRSTLVGKIQVGEYSSIIGLIKRTQRIYGNVFTVSGVIAAFRRSALAEVGYWSDDMITEDIDISWKLQLNQWTIFYEPRALCWILMPETLKGLWKQRLRWAQGGAEVFLKNMTRLWRKENFRMWPLFFEYCLTTIWAFTCLVGFIIYAVQLAGVPLNIELTHIAATHTAGILLCTLCLLQFIVSLMIENRYEHNLTSSLFWIIWFPVIFWMLSLATTLVSFTRVMLMPKKQRARWVSPDRGILRGMNNL

SEQ ID NO: 7 - DNA, 30 bp, Escherichia coli
tacagttaagtgtgttatcggtgcagagcc

SEQ ID NO: 8 - DNA, 31 bp, Escherichia coli
ctcaacgcctggctgattaaaccaactattc

SEQ ID NO: 9 - DNA, 7500 bp, Escherichia coli
atgtattcaagtagcagaaaaaggtgcccgaaaaccaaatgggctttgaaacttcttactgccgcatttttagcagcgagtcccgcggcgaagagtgctgttaataacgcctatgatgcattgattattgaagctcgcaagggtaatactcagccagctttgtcatggtttgcactaaaatcagcactcagcaataaccaaattgctgactggttacagattgccttatgggccgggcaagataaacaggttattaccgtttacaaccgctaccgtcatcagcaattaccagcgcgtggttatgcagctgtcgccgtcgcttatcgtaacctgcaacaatggcaaaactcgcttacactgtggcaaaaggcgctctctctggagccgcaaaataaggattatcaacggggacaaattttaaccctggcagatgctggtcactatgatactgcgctggttaaacttaagcagcttaactctggagcaccggacaaagccaatttactcgcagaagcctatatctataaactggcggggcgtcatcaggatgaattacgggcgatgacagagtcattacctgaaaatgcatctacgcaacaatatcccacagaatacgtgcaggcattacgtaataatcaacttgctgccgcgattgacgatgccaatttaacgccagatattcgcgctgatattcatgccgaactggtcagactgtcgtttatgcctacgcgcagtgaaagtgaacgttatgccattgccgatcgcgccctcgcccaatacgctgcattagaaattctgtggcacgataacccagaccgcactgcccagtaccagcgtattcaggttgatcatcttggcgcgttattaactcgcgatcgttataaagacgttatttctcactatcagcgattaaaaaagacggggcaaattattccgccctgggggcaatattgggttgcatcggcttatctcaaagatcatcagccgaaaaaagcacagtcaataatgaccgagctcttttatcacaaggagaccattgccccggatttatccgatgaagaacttgcggatctcttttacagccacctggagagtgaaaattatccgggcgcgctaactgtcacccaacataccattaatacttcgccgcctttccttcggttaatgggcacgcctacgagcatcccgaatgatacctggttacaggggcattcgtttctctcaaccgtagcaaaatatagtaatgatcttcctcaggctgaaatgacagccagagagcttgcttataacgcaccaggaaatcagggactgcgcattgattacgcgagtgtgttacaagcccgcggttggcctcgtgcagcagaaaatgaattaaaaaaagcagaagtgatcgagccacgtaatattaatctggaggttgaacaagcctggacagcattaacgttacaagaatggcagcaggcagctgtcttaacgcacgatgttgtcgaacgtgaaccgcaagatcccggcgttgtacgattaaaacgtgcggttgatgtacataatcttgcagagcttcgtatcgctggctcaacaggaattgatgccgaaggcccggatagtggtaaacatgatgtcgacttaaccaccatcgtttattcaccaccgctgaaggataactggcgcggttttgctggattcggttatgccgatggacaatttagcgaaggaaaagggattgttcgcgactggcttgcgggtgttgagtggcggtcacgtaatatctggctcgaggcagagtacgctgaacgcgttttcaatcatgagcataaacccggcgcgcgcctgtctggctggtatgattttaatgataactggcgtattggttcgcaactggaacgcctctctcaccgcgttccattacgggcaatgaaaaatggtgttacaggcaacagtgctcaggcttatgttcgctggtatcaaaatgagcggcgtaagtacggtgtctcctgggctttcactgatttttccgacagtaaccagcgtcatgaagtctcacttgagggtcaggaacgcatctggtcttcaccatatttgattgtcgatttcctacccagtctgtattacgaacaaaatacagaacacgataccccatactacaaccctataaaaacgttcgatattgttccggcatttgaggcaagccatttgttatggcgaagctatgaaaatagctgggagcaaatattcagcgcaggtgttggtgcctcctggcaaaaacattatggcacggatgtcgtcacccaactcggctacgggcaacgcattagttggaatgacgtgattgatgctggcgcaacgctacgctgggaaaaacgaccttatgacggtgacagagaacacaacttatacgttgaattcgatatgacattcagattttaaggataaatatgttacgtaatggaaataaatatctcctgatgctggtgagtataattatgctcaccgcgtgcattagccagtcaagaacatcatttataccgccacaggatcgcgaatctttactcgccgagcaaccgtggccgcataatggttttgtagcgatttcatggcataacgttgaagacgaagctgccgaccagcgttttatgtcagtgcggacatcagcactgcgtgaacaatttgcctggctgcgcgagaacggttatcaaccggtcagtattgctcaaattcgtgaagcacatcgaggaggaaaaccgctaccggaaaaagctgtagtgctgacttttgatgacggctaccagagtttttatacccgcgtcttcccaattcttcaggccttccagtggcctgctgtatgggcccccgtcggcagttgggtcgatacgccagcggataaacaagtaaaatttggcgatgagttggtcgatcgagaatattttgccacgtggcaacaagtgcgagaagttgcgcgttcccggctcgttgagctcgcttctcatacatggaattctcactacggtattcaggctaatgccaccggcagcttattgcctgtatatgtaaatcgtgcatattttactgaccacgcacggtatgaaaccgcagcagaataccgggaaagaattcgtctggatgctgtaaaaatgacggaatacctgcgtacaaaggttgaggtaaatccacacgtttttgtttggccttatggcgaagcgaatggcatagcgatagaggaattaaaaaaactcggttatgacatgttcttcacccttgaatcaggtttggcaaatgcgtcgcaattggattccattccgcgggtattaatcgccaataatccctcattaaaagagtttgcccagcaaattattaccgtacaggaaaaatcaccacaacggataatgcatatcgatcttgattacgtttatgacgaaaacctccagcaaatggatcgcaatattgatgtgctaattcagcgggtgaaagatatgcaaatatcaaccgtgtatttgcaggcatttgctgatcccgatggtgatgggctggtcaaagaggtctggtttccaaatcgtttgctaccaatgaaagcagatatttttagtcgggttgcctggcaattacgtacccgctcaggtgtaaacatctatgcgtggatgccggtattaagctgggatttagatcccacattaacgcgagtaaaatacttaccaacaggggagaaaaaagcacaaattcatcctgaacaatatcaccgtctctctcctttcgatgacagagtcagagcacaagttggcatgttatatgaagatcttgccggacatgctgcttttgatggcatattgttccacgatgatgctttgctttcagattatgaagatgccagtgcaccggctatcacggcttatcagcaagcaggctttagcgggagtctgagcgaaattcgacaaaacccggagcaatttaaacagtgggcccgctttaaaagtcgtgcgttaactgacttcactttagaacttagtgcgcgcgtaaaagccattcgcggtccacatattaaaactgcacgaaatatttttgcacttccggtaatacaacctgaaagtgaagcctggtttgcacagaattatgctgatttcctaaaaagctatgactggaccgctattatggctatgccttatctggaaggtgtcgcagaaaaatcggctgaccaatggttaatacaattgaccaatcaaattaaaaacatccctcaggctaaagacaaatctattttagaattacaggcacaaaactggcagaaaaatggtcagcatcaggctatttcttcgcaacaactcgctcactggatgagcctattacaactgaatggagtgaaaaactatggttattatcccgacaattttctgcataaccaacctgaaatagaccttattcgtcctgagttttcaacagcctggtatccgaaaaatgattaatcgcatcgtatcgttttttatattatgtctggtgttatgcatacccctatgcgtagcgtactttcactctggtgaactgatgatgaggttcgttttcttctggccgttttttatgtccattatgtggattgttggcggcgtctatttctgggtctatcgtgaacgccactggccgtggggagaaaacgcaccagctccccagttgaaagataatccgtctatctccattatcattccctgttttaatgaggagaaaaacgttgaggaaaccatacacgccgctttagcacagcgttatgagaacattgaagttattgccgtaaatgacggttcaacagataaaacccgtgccatcctggatcgcatggctgcacaaattccccatttgcgggtcattcatctggcgcaaaaccaggggaaagccattgcgcttaaaaccggagctgccgcggcgaaaagtgaatatctggtgtgcattgatggcgatgcgttattagaccgcgatgcggcggcatatattgtggaaccgatgttgtacaacccgcgtgtgggtgccgtaaccggtaatcctcgtattcgaacacgttctaccctggtgggtaaaattcaggttggcgagtattcctcaattattggtttgatcaagcgaacccagcgtatctatggaaacgtatttaccgtttccggtgttattgccgcatttcgtcgcagcgccctggcagaagtgggttactggagtgacgatatgatcaccgaagatattgatattagctggaagctgcagttgaatcagtggacgattttttacgagccacgggcactgtgctggatattaatgcctgaaacgttaaaagggctgtggaaacagcgcctgcgctgggctcagggcggtgcagaagtattcctcaaaaatatgacaaggttgtggcgcaaagaaaactttcgaatgtggccgctgttttttgaatactgcctgacgacaatatgggccttcacctgcctggtcggtttcattatttacgcagtccaacttgccggtgtaccgttaaatattgaattgacacatatcgctgcgacacatactgccggaatattattgtgtacgttatgtttactgcaatttattgtcagcctgatgatcgagaatcgctatgagcataatctgacttcatcgcttttctggattatttggttcccggttattttctggatgctgagcctggcaacgacattggtatcatttacacgagtcatgttgatgcctaaaaagcaacgcgcccgttgggtaagtcccgatcgcgggattctgagaggttaatatgaacaatttaattattacgacccgacaatcaccagtacgtttactggttgattatgttgccacaaccatcttgtggacattatttgcgttgttcatattcttattcgccatggatctgctgacgggttattactggcaaagcgaggccagaagccgacttcagttctattttttgctggcagtggcgaatgccgtcgtgttaattgtctgggcgctgtacaataagctgcgttttcaaaaacagcagcatcatgcagcctaccaatatacgccgcaagaatatgcagagagcttagcaatacctgatgagctctatcagcaactacaaaaaagccacaggatgagcgtacacttcaccagccaggggcaaataaaaatggttgtttcagaaaaagcgctagtccgggcataaacacccaaaacaaagcccggttcgcccgggctctgcaccgataacacacttaactgtaggcatgcagcgtacgttggcaaagtgccgaacgtacgcagtcctctttaccgaaccggacgatcccaaccatttcatcttcttcgaaacgttccagcgcgtcacttaatccggagcacacgccgcgaggcaaatcgcattgcgtgatatcaccgttgacgataaccgtcacgttctccccgaggcgggttaaaaacattttcatttgcgcggcagtcacattctgcgcctcgtcaagaatgacgactgcattttcaaaggtacgtccacgcatataggcgaacggcgcaatttccaccttccctatttccggtcgcaggcagtactgcataaaggaagcccctaagcgccggaccagcacgtcgtagaccgggcgaaaatagggagcaaacttttctgcgatatctccaggtaagaagccaagatcttcatcggcttgcagaactggacgggtgacgataatcctgtcgacatccttatgtatcagggcctctgccgcttttgctgcgctgatccaggtttttccgcacccggcttcgcccgtggcgaatatcagctgcttactctcaatagccttcagatagtgcaattgcgcttcatttcgcgcgaggatgggcgaagtatcgcgactgtcgcgggccataccaatggcttctacgccgcccatctgcacaagcgaggtgaccgattcttcttcacgctgcttatggctgcgcgaatcccgtctcagcacacgttttgcctcgcgacgagctttgatcactgctttttgtcttcccatggagagcaccttgagttgtttgtattcatcacacgcgccgttggcagcgcgattatgcgcacgaacatcagagggttggcttccttgtaagccatagtttgcttttggataaaatgccgaaaaacggctacgcgcaccgtttacggcgtcggtaacacatgaaaagaaaggatgaggttgaaaatgcaaagtgacgagatgactaccggaggagaaaactccgcgagtggtggcgcgttgattatctaaaacatgtccagtacaggacgttaccatccgcgatctccatagtgactgactatcactgccgggaacttccgctgctacttaataagtacaacagatctcgcatttattgcaacaatatatttacttatatttaactataaaacaccatttcagtgacattagtttctactggaaagatgacagagtgatgacagtgatgaaaaaagctgtgtgctttcagcaggatttg

(19) United States
(12) Patent Application Publication
(10) Pub. No.: US 2004/0116371 A1
(43) Pub. Date: Jun. 17, 2004

(54) Methods for polysaccharide adhesion synthesis modulation

(76) Inventors: Tony Romeo, Decatur, GA (US); Xin Wang, Atlanta, GA (US)

Correspondence Address: MERCHANT & GOULD PC, P.O. BOX 2903, MINNEAPOLIS, MN, 55402-0903, US

(21) Appl. No.: 10/675,226

(22) Filed: Sep. 29, 2003

Related U.S. Application Data
(60) Provisional application No. 60/414,352, filed on Sep. 30, 2002.

(51) Int. Cl. A61K 48/00

(52) U.S. Cl. 514/44

(57) ABSTRACT

There is provided a method for modulation of polysaccharide adhesin synthesis involving products of the ycdSRQP gene operon in bacteria, depicted in SEQ. ID. NO. 1 and 2. Also provided is the use of an inhibitor of a product of the ycdSRQP operon in improving the response of a mammalian patient suffering from a bacterial infection.

### CROSS-REFERENCE TO RELATED APPLICATION

This application claims priority from U.S. provisional application No. 60/414,352, filed Sep. 30, 2002, which is pending.

### FIELD OF THE INVENTION

The invention relates to methods for polysaccharide adhesin modulation and particularly adhesin synthesis relating to biofilm formation.

### BACKGROUND OF THE INVENTION

Microorganisms commonly attach to living and nonliving surfaces, including those of indwelling medical devices, and form biofilms made up of extracellular polymers. In this state, microorganisms are highly resistant to antimicrobial treatment and are tenaciously bound to the surface. Biofilms represent a distinct physiological state, designed to provide a protected environment for survival under hostile conditions. Many chronic infections that are difficult or impossible to eliminate with conventional antibiotic therapies are known to involve biofilms. A partial list of the infections that involve biofilms includes: otitis media, prostatitis, vascular endocarditis, cystic fibrosis pneumonia, meliodosis, necrotizing faciitis, osteomyelitis, peridontitis, biliary tract infection, struvite kidney stone and host of nosocomial infections.

Biofilm formation is a two-step process that requires the adhesion of bacteria to a substrate surface followed by cell-to-cell adhesion, forming the multiple layers of the biofilm. Bacterial or microorganism adherence is thought to be the first crucial step in the pathogenesis and biofilm formation. A number of factors influence an organism's ability to adhere to a surface. The early stages of adherence are influenced by non-specific forces such as surface charge, polarity and hydrophobic interactions. Later stages of adherence are thought to involve more specific interactions between adhesins and receptors. Studies on the adherence of bacteria to a biotic or abiotic surface are focused in part on the role of the extracellular polysaccharide or glyocalyx, also known as slime. Currently, extracellular polysaccharide is thought to play a role in the later stages of adherence and persistence of infections. It may serve as an ion-exchange resin to optimize a local nutritional environment, prevent penetration of antibiotics into the macrocolony, and protect bacteria from host defense mechanisms. Extracellular polysaccharide appears in the later stages of attachment and is not present during the initial phase of adherence. However, study of exopolysaccharide has lended little to prevention of initial adherence by the bacteria.

Several studies have examined biofilm components and/or genetic factors in biofilm formation.

Potential adhesins in bacteria such as Staphylococcus epidermidis have been identified, including the polysaccharide adhesin (PS/A). PS/A contains a complex mixture of monosaccharides and purified PS/A blocks adherence of PS/A producing strains of S. epidermidis. It appears that PS/A and SAA (slime associated antigen) are distinct. It has been hypothesized that each functions in different stages of the adherence process with one or more of these adhesins responsible for initial attraction while others are needed for aggregation to form the macrocolonies.

The polysaccharide intercellular adhesin (PIA) is composed of linear β-1,6-linked glucosaminylglycans in Staphylococcus epidermidis and Staphylococcus aureus. Mack, D., et al., J. Bacteriol., 178: 175-183 (1996); Crampton, S. E., et al., Infect. Immun., 67: 5427-5433 (1999).

Polymeric β-1,6-N-acetylglucosamine has only been reported in Staphylococci. No such polymer is believed to have been previously reported in any gram-negative species.

Genetic factors in biofilm formation have been considered for Staphylococci (Gerke, J. Biol. Chem., 273: 18586 (1998)) and Yersinia pestis (Hare, J. Bacteriol., 181:4896 (1999)).

Studies by others have failed to provide substantive evidence of unique metabolic requirements for biofilm formation.

Other microbial adhesins have been reported. Such adhesins include: polysaccharide antigen from Pseudomonas aeruginosa slime (U.S. Pat. No. 4,285,936; U.S. Pat. No. 4,528,458); Escherichia coli fimbrial protein adhesins (Orskov, I., et al., Infect. Immun., 47: 191-200, 1985; Chanter, H., J. Gen. Microbiol. 125: 225-243 (1983) and Moch, T., et al., Proc, Natl, Acad, Sci., 84: 3462-3466 (1987)); lectin-like glycoprotein adhesin (Bacteroides fragilis group); a 70 kDa adhesin (Rogemond, V., et al., Infect. Immun., 53: 99-102 (1986)); and, uroepithelial cell adhesin protein of 17.5 kDa (Proteus mirabilis) (Wray, S. K., et al., Infect. Immun., 54: 43-49 (1986)).

Crude extracellular products from the slime of homologous strains of Staphylococcus epidermidis inhibit the adherence of homologous bacterial cells to polymeric materials used as catheters and prostheses. Materials derived from the surface of such cells have been used as vaccines to produce antibodies directed against homologous bacteria. For example, Frank (French Patent Application 85-07315, Nov. 21, 1986); Pier, (U.S. Pat. No. 5,055,455 Oct. 8, 1991; U.S. Pat. No. 4,443,549; U.S. Pat. No. 4,652,498); and McKenny (Canadian Pat. No. CA2,333,931, Jan. 12, 2001).

The complete genome of E. coli K12 was reported by Blattner (Science 277: 1453 (1997). However, this report failed to suggest any function for the region encoding the ycdSRQP operon. Information is also provided in Hare, J. M. and McDonough, K. A., J. Bacteriol. 181: 4896-4904 (1999).

Thus, it is an object of the invention to provide an improved method for polysaccharide adhesin modulation.

### SUMMARY OF THE INVENTION

An embodiment of the invention provides, inter alia, the ycdSRQP operon, products thereof and methods and uses therefore. This operon was identified by independent insertions in ycdS (SEQ ID NO: 1), ycdR (SEQ ID NO: 2) and ycdQ (SEQ ID NO: 3), which severely decreased biofilm formation in E. coli wild type strain MG1655.

YcdQ of E. coli appears to be associated with the inner membrane and contains 5 putative membrane-spanning domains. YcdR appears to have a function as a polysaccharide deacetylase. YcdR is also believed to be involved in the transport of PIA. YcdR is believed to be a lipoprotein in its active form. YcdS of E. coli is a putative outer membrane protein believed to be involved in the extracellular localization/transport of the PIA polymer and/or as a docking protein to assist in the formation of an intercellular bridge between cells.

An embodiment of the invention provides ycdS, ycdR and ycdQ polynucleotides and polypeptides and uses and methods relating thereto.

While the invention is not limited to any particular mechanism of action, it appears that the genes of this operon are involved in the production and biological function of a linear β-1,6-N-acetylglucosamine polymer that functions as an adhesin in biofilm formation. Biofilm formation is believed to depend on the production of a polysaccharide intercellular adhesin (PIA). The PIA represents and mediates the intercellular adherence of bacteria to each other and accumulation of a multilayered biofilm.

TABLE 1
Metabolic Conversion of Glycogen to PIA in E. coil

| Steps | Gene products |
|---|---|
| 1. Glycogen → Glucose-1-Phosphate | GlgP, GlgX |
| 2. Glucose-1-Phosphate → Glucose-6-Phosphate | Pgm |
| 3. Glucose-6-Phosphate → Fructose-6-Phosphate | Pgi |
| 4. Fructose-6-Phosphate → GlcN-6-P | GlmS |
| 5. GlcN-6-P → GlcN-1-P | GlmM |
| 6. GlcN-1-P → GlcNAc-1-P | GlmU |
| 7. GlcNAc-1-P → UDP-GlcNAc | GlmU |
| 8. UDP-GlcNAc →β-1,6-GlcNAc (n + 1) | YcdQ |

Table 1. Pathway for converting glycogen into PIA in E. coli. GlgX is the glycogen debranching enzyme, which hydrolyzes the 1,6-linkages of glycogen, and thereby enhances the conversion of glycogen to glucose-1-phosphate by glycogen phosphorylase (GlgP). GlmU is required to both the aceylation of GlcN-1-P and the UDP-GlcNAc pyrophosphorylase reaction.

In an embodiment of the invention there are provided products of the ycdSRQP operon.

In an embodiment of the invention there is provided a method of identifying inhibitors of products of the ycdSRQP operon.

In an embodiment of the invention there is provided a method of decreasing biofilm formation by biofilm-forming bacteria by decreasing expression of one or more products of the ycdSRQP operon.

In an embodiment of the invention there is provided the use of a product of the ycdSRQP operon to modulate polysaccharide adhesin synthesis.

In an embodiment of the invention there is provided the use of a product of the ycdSRQP operon to modulate biofilm formation.

In an embodiment of the invention there is provided use of a product of the ycdSRQP operon in improving the response of a mammalian patient suffering from a bacterial infection by biofilm forming bacteria.

In an embodiment of the invention there is provided a method of inhibiting polysaccharide deacetylation by reducing YcdR activity.

In an embodiment of the invention there is provided a method of inhibiting adhesin transport by reducing YcdR activity.

In an embodiment of the invention there is provided a method of reducing extracellular adhesin binding in E. coli by reducing YcdS activity.

In an embodiment of the invention there is provided a method of improving the response of a mammalian patient suffering from a bacterial infection to antibiotics for treatment of said bacterial infection comprising reducing biofilm formation by infecting the bacteria.

In an embodiment of the invention there is provided a method of facilitating the reduction of bacterial load in a mammalian patient suffering from bacterial infection, comprising inhibiting the activity of a product of the ycd operon in at least some of the infecting bacteria.

In an embodiment of the invention there is provided a method of decreasing cell to cell biofilm links by reducing YcdS activity.

In an embodiment of the invention there is provided a method of reducing adhesin synthesis in E. coli by reducing YcdQ activity.

In an embodiment of the invention there is provided a method of reducing 13-1,6-N-acetylglucosamine (13-1,6Glc NAc) polymer synthesis by reducing YcdQ activity.

In an embodiment of the invention there is provided a method of reducing glycosyltransferase activity in E. coli by reducing YcdQ activity.

In an embodiment of the invention there are provided antibodies to E. coli β-1,6Glc NAc.

In an embodiment of the invention there is provided a use and method of using antibodies to E. coli β-1,6Glc NAc in an assay to identify biofilm production and an assay to identify biofilm reduction.

In an embodiment of the invention there is provided a method of reducing biofilm formation by reducing the activity of YcdQ in a plurality of bacterial cells.

In an embodiment of the invention there is provided a method of reducing biofilm formation by reducing the activity of YcdS in a plurality of bacterial cells.

In an embodiment of the invention there is provided a method of reducing biofilm formation by reducing the activity of YcdR in a plurality of bacterial cells.

In an embodiment of the invention there is provided a method of reducing biofilm formation by reducing the activity of YcdP in a plurality of bacterial cells.

There are provided products of the ycdSRQP operon and uses and methods for using these products in the production of antibodies to the products of these genes. These antibodies may be useful diagnostically in identifying aberrations in proteins encoded by this operon and therapeutically to reduce cell-cell interactions mediated by these products of the ycdSRQP operon, and particularly YcdS. Additionally, these gene products may be used in screening tests for inhibitors of these products.

There is provided a method of identifying inhibitors of products of ycdSRQP operon comprising selecting a gene product of interest, assaying the activity of that gene product under control conditions, adding a potential inhibitor of the gene product, assaying the activity of the gene product in the presence of the potential inhibitor, and ascertaining whether the presence of the potential inhibitor resulted in an inhibition of the function of that gene product.

There is provided a use and a method of decreasing biofilm formation. This may be accomplished by a variety of means, including using antisense RNA sequences to decrease expression of the products of the genes of ycdSRQP operon.

There is provided a use and a method of using antisense sequences to genes, or portions thereof, of the ycdSRQP operon to reduce the rate of conversion of UDP-GlcNAc to β-1,6GlcNAa polymeric units in an E. coli containing environment. This may be accomplished by reducing the expression or activity of one or more genes of the ycd operon involved in biofilm formation. For example, antisense sequences complementary to mRNA encoding YcdS or YcdQ may be employed to reduce translation of the corresponding protein, and thus the activity of that protein.

Antisense sequences may be administered exogenously in bacterial culture, by administration to a patient suffering from E. coli infection, or by gene therapy to introduce genetic material encoding the antisense sequence directly into E. coli, and/or into the patient in a form which it can be excreted from the cell, and taken up by the invading E. coli.

In some instances, the bacteria is at least one of E. coli or Staphylococcus.

In some instances, the E. coli is E. coli K12.

In some instances, the E. coli is any member of the E. coli species.

### BRIEF DESCRIPTION OF THE DRAWINGS

FIG. 1 is a graph showing plasmid clones (pUCPGA372) stimulate biofilm formation in a variety of E. coli strains. Bar graph A shows the effects in MG1655 for various isogenic strains represented by bars 1 to 7. Bar graph B shows the effects of ycd genes in TRMG1655 (csrA::kanrR) for various strains represented by bars 1 to 7.

FIG. 2 is a graph showing the fractionation of polysaccharide adhesion by gel filtration FPLC, cell extract from strain TRMG1655 cpsE ycdQ containing pUCPG372 (graph A) or pUC19 (graph B).

### DETAILED DESCRIPTION OF THE INVENTION

### EXAMPLE 1

### Molecular Cloning of ycd Operon

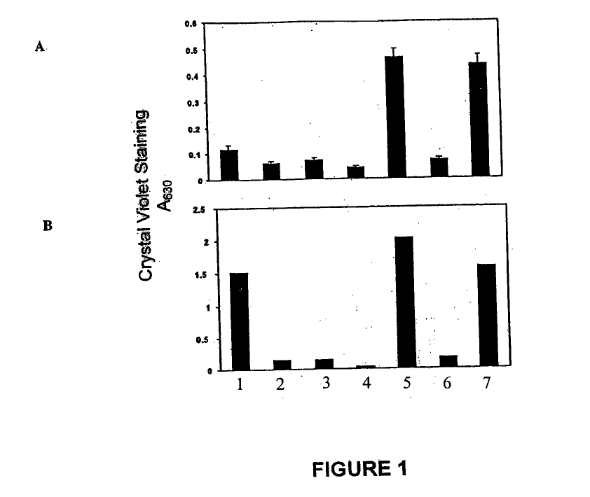

Plasmid clones (pUCPGA372) of this operon complement ycdQ and ycdS mutations and stimulate biofilm formation in a variety of E. coli strains. FIG. 1 shows the effect of ycd genes on biofilm formation. Bar graph A shows the effects in MG1655. Isogenic strains represented by bars 1 to 7 are MG1655. ycdQ mutant, ycdS mutant, ycdQ mutants containing pUC19 or pUCPGA372 (cloned ycdSRQP) and ycdS mutant containing pUC19 or pUCPGA372, respectively. Bar graph B shows the effects of ycd genes in TRMG1655 (csrA::kanrR). Strain identities for bar 1 to 7 are TRMG1655, ycdQ mutant, ycdS mutant, ycdQ mutants containing pUC19 or pUCPGA372, and ycdS mutant containing pUC19 or pUCPGA372, respectively.

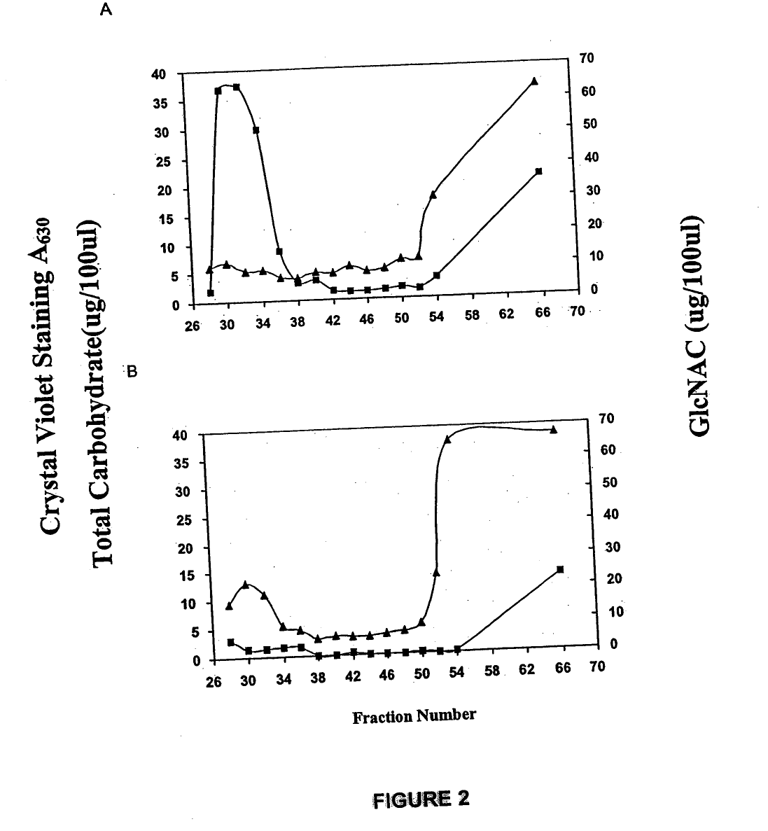

A purification protocol was designed, which yielded a highly enriched polymeric GlcN fraction from a strain containing the ycdSRQP plasmid clone. FIG. 2 shows the fractionation of polysaccharide adhesion by gel filtration FPLC. Cell extract from strain TRMG1655 cpsE ycdQ containing pUCPG372 (graph A) or pUC19 (graph B) was fractionated using a Sephacryl S-200 (16/60) column. Fractions of 2 ml were collected and analyzed for total carbohydrate (triangle) and, after hydrolysis, for glucosamine (square). The straight line on each of graph A and B indicates the void volume of the column and was determined using 2-MDa blue dextran.

The polysaccharide was used for routing polyclonal antibody production and for affinity-column purification of the antibodies.

The antisera are used to develop a simple quantitative assay for the polymer, including ELISA. There is a correlation between ycd gene expression, β-1,6GlcNAc synthesis, and biofilm formation in E. coli.

Mutations in Cloned ycd Operon Carried by pUCPGA372.

The ycd genes were cloned and were found to differ from the sequence reported by Blattner as follows.

In the ycdR gene, nucleotide 723 was changed from A to G, and the codon was changed from GTT (Leu) to GCT (Ser). Two other mutations in ycdS gene, in which nucleotide 582 and 389 were changed from T to C, and the codons were changed from TAA (Asn) to TAG (Asp), and AAC (Gln) to AGC (Arg) respectively.

With reference to SEQ ID NO: 6, the numbering for the full DNA sequence of ycdS starts at the A of the ATG initiation codon. Individual mutations are numbered from the start codons of each gene. In SEQ ID NO: 6, underlining indicates codons affected by point mutations and the insertion sites for the various transposon mutants are shown by downward facing arrows.

### EXAMPLE 2A

### Involvement of yCdSRQP Operon in the Biosynthesis of Unbranched β-1,6-GlcNAc (Polysaccharid Intercellular Adhesin)

The ycdSRQP operon, which encodes proteins needed for the production and function of a biofilm polysaccharide adhesin, was cloned and sequenced, and mutants were prepared.

Methods:

Plasmid Construction. The ycd operon was amplified by polymerase chain reaction from chromosomal DNA of MG1655 using the oligonucleotide primers TACAGTTMGTGTGTTATCGGTGCAGAGCC (SEQ ID NO: 4) and CTCMCGCCTGGCTGATTAAACCMCTATTC (SEQ ID NO: 5). The PCR product, a 6.9 kb fragment, was purified by QIAquick Gel Extraction Kit (QIAGEN) and cloned into vector pCR-XL-TOPO (Invitrogen) using D H5α as the host for transformation. Approximately 120 clones were screened for increased biofilm production. One clone pCRPGA37, increasing biofilm ˜6-fold when expressed in DH5α was subsequently treated with HindIII and Xbal, and the insert DNA was subcloned into pUCl9 to yield plasmid pUCPGA372. PCRPGA37 was sequenced.

Transposon Mutagenesis. Transposon mutants were generated by infecting TRMG1655ΔfimB-HΔmotB with λNK1324 at a multiplicity of infection of 0.2, essentially as described in Romeo et al., J. Bacteriol. 175: 4744 (1993) and Kleckner, Meth. Enzymol. 204:139 (1991). The insertion mutants were selected on Kornberg agar containing 30 ug/ml chloramphenicol. Chloramphenicol-resistant colonies were picked and grown at 26° C. in 96-well, polystyrene microtiter plate containing CFA with 30 ug/ul chloramphenicol. After 24 hr, the cells were subculture into corresponding wells in 96-well microtiter plates containing CFA with 30 ug/ul chloramphenicol and incubated at 26° C. for 24 hr. Turbidity in the wells was determined to avoid isolation of mutants with growth defects, and biofilm by the mutants was measured. Mutants with altered ability to form biofilms were saved. These candidate mutants were streaked to isolate single colonies on Kornberg agar and retested for their ability to form biofilm. Candidate insertion mutations were transferred by P1vir transduction into the original parent strain or related strains and retested for the biofilm development. Stock cultures were saved at −80° C.

Purification of the Polysaccharide Adhesin. E. coli strains containing pUCPGA372 or pUCl9 were grown for 24 hours at 37° C. with shaking at 250 rpm in CFA medium containing 100 μg/ml ampicillin. Bacterial cells were harvested and resuspended in 50 mM Tris.HCl (pH 8.0). Cell extracts were prepared by lysozyme-EDTA treatment in the presence of DNase, RNase and α-amylase (Sigma) and were phenol extracted (Wolf-Watz, H., J. Bacteriol., 115: 1191-1197, 1973; Westphal, O. and Jann, K., J., Methods Carbohyd. Chem., 1964). The aqueous phase was extracted with chloroform, concentrated in an Amicon cell with a YM10 membrane and fractionated by FPLC on Sephacryl 5-200. The column was equilibrated with 0.1 M PBS (pH 7.4) and eluted with the same buffer. The GlcNAc-containing polysaccharide was detected by the MBTH assay following hydrolysis for 2 hours at 110° C. in 0.5M HCl (Smith, R. L. and Gilkerson, E., Anal. Biochem., 98: 478-480, 1979). Total carbohydrate was measured by phenol-sulfuric acid assay (Dubois, M., et al., Anal. Chem. 28: 350-356, 1959).

Quantitative Biofilm Assay. Bacterial overnight cultures were inoculated 1:100 dilution into 96-well microtiter plate containing 200 □l/well fresh medium plus appropriate antibiotics. The plates were incubated at 26° C. for 24 hours. Biofilm was measured by discarding the medium, rinsing the wells with water (three times), and staining bound cells with crystal violet (BBL). The dye was solubilized with 33% acetic acid, and absorbance at 630 nm was determined using a microtiter plate reader. Background staining was corrected. All comparative analyses were conducted by incubating strains within the same microtiter plate to minimize variability. Each experiment was performed at least in triplicate.

### EXAMPLE 2B

### Precursor-Product Relationship of Glycogen to PIA by ¹³C NMR

Direct evidence for the precursor-product relationship of glycogen to PIA is established using ¹³C glucose pulse labelling at the transition to a stationary phase. During this time, replication and growth decline, while glycogen synthesis remains active. Thus, ¹³C incorporation into glycogen is efficient. NMR spectra of growing cultures are monitored in real time for glycogen and PIA. The availability of a strain disrupted in YcdQ is a powerful asset for these studies, and allows the precursor-product relationship to be firmly established. YcdQ blocks PIA synthesis, but not glycogen synthesis. Glucose differentially labeled in carbons 1, 2 or 6 is used to follow the conversion to glycogen and PIA. The commercial availability of these substrates allows monitoring of bacterial metabolism.

### EXAMPLE 3

### YcdQ for the Cell-Free Synthesis of Poly B-1,6-GlcNAc (PIA)

To assess the potential role of ycdQ and the other ycd genes in synthesis of β-1,6-GlcNAc, membranes are prepared from wild type and nonpolar mutants, incubated with UDP-N-acetyl-D-[U-14C] glucosamine. The resulting oligosaccharides are separated by thin-layer chromatography and detected by autoradiography (Gerke, C et al, J. Biol. Chem. 273: 18586-18593, 1998). YcdQ is a N-acetylglucosamine transferase which adds N-acetylglucosamine to the growing polymer. Thus, YcdQ is very important for cell-free synthesis of PIA, although other ycd genes can affect the reaction rate and/or extent of the polymerization reaction.

### EXAMPLE 4

### The Roles of ycd Genes in PIA Transport and PIA-Dependent Adhesion

There is a mechanism by which PIA traverses the outer membrane of E. coli. In some instances, YcdS is involved in PIA export. To show this, PIA is synthesized in isolated membranes from an ycdS nonpolar mutant. This PIA is detectable in cell lysates, but is not found on the cell surface using antibody binding to whole cells. YcdS is involved in the formation of cell to cell biofilm links. In some instances YcdS also plays a role as an anchor protein that helps to attach PIA to the cell surface. In such instances, significant amounts of PIA are observed in extracellular fractions, but little cell bound materials is present.

YcdR plays a role in polysaccharide deacetylation. This is evaluated by NMR studies. The role of YcdR in transit is proven by immunolocation studies.

YcdQ is involved in adhesin synthesis. This is shown by the reduction of biofilm formation following disruption of the ycdQ gene.

Thus, the invention provides, in one embodiment, a mutation of the ycdR gene, sufficient to alter YCdR activity: The mutation is a non-conservative mutation, disrupting expression of the normal gene product. In some instances the mutation changes the encoded amino acid from an aliphatic amino acid to a hydrophilic amino acid. In some instances the mutation enables the encoded amino acid to engage in hydrogen bonding, which the wild type encoded amino acid was unable to engage in. In some instances the mutation is a frame shift mutation resulting in a loss of the downstream encoded gene product. In some instances the mutation introduces a stop codon into the gene prior to the normal stop position, resulting in a truncated gene product.

In an embodiment of the invention there are provided non conservative mutants, of the ycdS gene.

In some instances, the mutation in ycdS gene is a non-conservative mutation resulting in coding for an uncharged amino acid (at physiological pH) where a charged amino acid appears in the wild type. In some instances, the mutation results in the replacement of a negatively charged amino acid with an uncharged amino acid (at physiological pH). In some instances, the mutation results in the replacement of an amino acid generally uninvolved in hydrogen bonding, with one capable of forming a hydrogen bond at physiological pH. In some instances the mutation is a frame shift mutation resulting in a loss of the downstream encoded gene product. In some instances the mutation introduces a stop codon into the gene prior to the normal stop position, resulting in a truncated gene product.

In some instances, the mutation in the ycdS gene results in the replacement of an uncharged amino acid (at physiological pH) with a charged amino acid. In some instances, this mutation results in the replacement of an uncharged amino acid with a positively charged (at physiological pH) amino acid. In some instances, the mutation results in the replacement of an amino acid having a side chain capable of acting as a hydrogen bond acceptor with an amino acid incapable of acting as a hydrogen bond acceptor (at physiological pH).

Mutation of the YcdP gene substantially prevents biofilm formation. Thus, YcdP is needed for biofilm formation.

### EXAMPLE 5

### Inhibition of Biofilm Formation Through Interference with the Activity of Proteins Encoded by the ycd Operon

YcdQ is involved in the polymerization of UDP-N-acetylglucosamine to form β-1,6-N-acetylglucosamine polymer known as PIA (polysaccharide intercellular adhesin) from UDP-N-acetylglucosamine, which is required for biofilm formation.

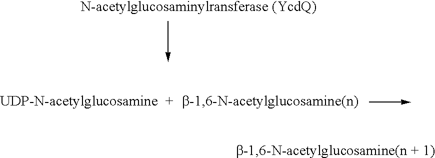

Crude Enzyme Preparation:

Crude membrane-bound N-acetylglucosaminyltransferase is prepared from overproducing strain of E. coli according to the method, described by Gerke, et al. (J. Biol. Chem., 273: 18586-18593, 1998). The overnight culture of E. coli is harvested by centrifugation, and the cell pellets, are resuspended in buffer A (50 mM Tris HCL pH 7.5, 10 mM MgCl₂ and 4 mM dithiothreitol; 2 μl/mg of cell wet weight). Grinding in a mortar disrupts DNase 1 (20 μg/ml) is added before breaking the cells. Unbroken cells are sedimented (2000×g, 10 min and the supernatant is saved. The procedure is repeated one to three times and all the supernatants are pooled. Membranes are sedimented from the crude extract by ultracentrifugation (200,000×g, 20 min) and resuspended in buffer A at a protein concentration of 5 mg/ml (5-fold concentration of the membrane proteins over the crude extract). For further purification, the crude membranes are extracted with 2% (w/v) Triton X-100 (in buffer A) for 2 h with gentle shaking, sedimented again, washed once with buffer A, and resuspended in the same volume of buffer A as the crude membranes. Protein concentration is determined by the method Bradford (Anal. Biochem., 72: 248-254, 1976).

Enzyme Assay:

In vitro reactions to analyze N-acetylglucosaminyltransferase activity are performed by incubating crude extracts with 0.4 mM UDP N-acetylglucosamine. In vitro synthesis of peptidoglycan is repressed by adding 50 μg/ml D-cycloserine (Lugtenberg, et al., J. Bacteriol., 109: 326-335,1972). For radiolabeling, 10 μM UDP-N-acetyl-D-(U-¹⁴C) glucosamine is added. Analytical mixture is carried out in a total volume of 50 μl. Reaction mixture is incubated for 12 h at 20° C. The reaction is stopped by the addition of 200 μl of water and boiling for 3 min. After centrifugation, the supernatant is loaded on a Sephadex A-25 anion-exchange column (gel volume, 300-500 μl) equilibrated with water. The column is washed with 2 ml of water. The unbound fraction (flowthrough and wash) is lyophilized. Radioactive products purified by Sephadex A-25 are subjected to gel filtration on a Bio-Gel P-2 column (90×1.5 cm) equilibrated with 0.1 M pyridine acetate (pH 6) at a flow rate of 0.3 ml/min. Fractions of 2 ml are collected and radioactivity is measured by liquid scintillation counting (Geremia, et al., Proc. Natl. Acad. Sci., USA., 91: 2669-2673,1994).

Identification and Selection of Enzyme Inhibitors

For all ycd proteins of interest, combinatorial libraries are screened to identify inhibitors. In addition, known inhibitors of key enzymes are tested using appropriate concentrations as reported in the literature. These inhibitors include natural or synthetic compounds and some analogues. These compounds are obtained from routine suppliers of reagent grade chemicals. The compounds showing maximum inhibition will be selected for determining their antibiofilm activity. Alternatively or additionally, libraries of compounds are tested for antibiofilm activity. Antibiofilm activity can include inhibiting YcdQ activity acid inhibiting biofilm formation by an E. coli culture.

Known deacetylase inhibitors and variants of such inhibitors are used to study their inhibitory effects on YcdR.

Short oligosaccharides of beta-1,6-GlcAc and synthetic/semisynthetic compounds capable of binding YcdS under physiological conditions are used to study their inhibitory effects on YcdS.

Known glycosyltransferase inhibitors, such as tunicamycin, bacitracin, isofagomine and azafagomine are used to study their inhibitory effects on N-acetylglucosaminyltransferase (YcdQ). In addition, variants of such inhibitors are examined. (For example, having acyl substitutions of a different size or having one or more altered or additional side groups.) N-acetylglucosaminyltransferase in a crude extract is incubated with different concentrations of inhibitors in the presence of 0.4 mM UDP-N-acetylglucosamine. In vitro synthesis of peptidoglycan is repressed by adding 50 μg/ml D-cycloserine (Lugtenberg, et al, J. Bacteriol., 109: 326-335, 1972). For radiolabeling, 10 μM UDP-N-acetyl-D-(U-¹⁴C) glucosamine will be added. The reaction is carried out in a total volume of 50 μl. The reaction mixture is incubated for 12 h at 20° C. The reaction is stopped by the addition of 200 μl of water and boiling for 3 min. After centrifugation, the supernatant is loaded on a Sephadex A-25 anion-exchange column (gel volume, 300-500 μl) equilibrated with water. The column is washed with 2 ml of water. The unbound fraction (flowthrough and wash) is lyophilized. Radioactive products purified by Sephadex A-25 a subjected to gel filtration on a Bio-Gel P-2 column (90×1.5 cm) equilibrated with 0.1 M pyridine acetate (pH 6) at a flow rate of 0.3 ml/min. Fractions of 2 ml are collected and radioactivity is measured by liquid scintillation counting (Geremia, et al., Proc. Natl. Acad. Sd., USA., 91: 2669-2673,1994).

Determining the Antibiofilm Activity of Selected Enzyme Inhibitors

The antibiofilm activity of selected enzyme inhibitors is evaluated using a microtiter plate format biofilm assay as described below. E. coli are used for biofilm inhibition assay. (The biofilm assay can be automated using robotics, if desired.) Further, the compounds showing significant antibiofilm activity are tested for their ability to block biofilm formation on commonly used medical devices.

Biofilm Assay:

Cultures of E. coli for biofilm assay are grown in Luria-Bertani (LB) at 37° C. Biofilm assays are carried out in colony-forming antigen (CFA) medium. Overnight cultures are inoculated 1:100 into fresh medium. In the microtiter plate assay, inoculated cultures are grown in a 96-well polystyrene microtiter plate for 24 h at 26° C. Growth of planktonic cells are determined by absorbance at 600 nm or total protein assay using a ELISA plate reader. Biofilm is measured by discarding the medium, rinsing the wells with water (three times), and staining bound cells with crystal violet (BBL). The dye is solubilized with 33% acetic acid, and absorbance at 630 nm is determined using a microtiter plate reader. For each experiment, background staining is corrected by subtracting the crystal violet bound to uninoculated controls. All comparative analyses are conducted by incubating 25 strains within the same microtiter plate to minimize the variability.

Biofilm Inhibition Studies:

At least two compounds from each enzyme inhibition study are selected for evaluation of their antibiofilm activity. The biofilm inhibition assay is performed for each compound. In the microtiter plate assay, inoculated cultures are grown in a 96-well polystyrene plate in the presence and absence (control) of selected enzyme inhibitors at different concentrations at 26° C. The plates are incubated for 24 h at 37° C. Biofilm is measured by discarding the medium, rinsing the wells with water (three times), and staining bound cells with crystal violet. The dye is solubilized with 33% acetic acid, and absorbance at 630 nm is corrected by subtracting the crystal violet bound to uninoculated controls. Each assay is performed 3-5 times. The concentrations of each enzyme inhibitor used for the assay is plotted against 0 D obtained for biofilm growth in order to indicate the percentage of inhibition in comparison with the control.

The compounds that inhibit biofilm formation on a microtiter plate are tested for their inhibitory effects on biofilm formation of E. coli in medical devices like urinary catheters.

The above methods are also applied, with suitable modifications employed in identifying, inhibitors of other products of the ycd operon, including YcdR and YcdS.

YcdR

In one approach, YcdR activity is determined by assaying the production of acetate from polysaccharide by HPLC. In one approach, radiolabeled PIA and its precursors are provided and the release of radiolabeled acetate is measured. Such release is proportional to YcdR activity.

### EXAMPLE 6

### Alternative Approach to Inhibitor Selection and/or Design

Method A:

(i) The proteins encoded by the genes of the ycd operon are purified by routine means, and their crystal structure is determined.

(ii) The structure of the region surrounding the amino acids in the YcdR which binds the polysaccharide is examined to identify the characteristics of molecules likely to interact specifically with that region.

(iii) Compounds having the general characteristics identified are screened for an ability to bind to the identified region in YcdR when immobilized in solution at physiological pH, tonicity and temperature.

(iv) Compounds showing an ability to bind to YcdR are identified. These compounds are, individually, added to E. coli cultures, and their effect on biofilm formation is determined.

Compounds capable of reducing biofilm formation in E. coli cultures are inhibitors of the YcdR protein.

Method B:

Steps (i) and (ii) of Method B are omitted.

(i) YcR is immobilized.

(ii) Large libraries of compounds are screened for an ability to bind to YcdR when immobilized.

(iii) Binding compounds are examined with respect to their ability to decrease biofilm formation in E. coli culture.

Either one of Method A or B is applied with suitable modification to identify inhibitors of YcdQ and YcdS. Modification will involve immobilizing the gene product of interest and, for Method A, step (ii), examining the structure of the region surrounding the amino acid by the codon containing a nucleotide mutation of which reduces biofilm formation an E. coli containing environment.

In some instances, inhibitors of products of the ycd operon may be encapsulated or otherwise treated to facilitate entry into E. coli cells, for example by liposome encapsulation including specific factors encouraging uptake by E. coli cells.

TABLE 2

Polynucleotide and Polypeptide Sequences of ycdS,
ycdR and ycdQ (Sequences from Escherichia coil).
(Note: Sequence numbering differs. Examples
and discussions refer to numbering of SEQ ID NO: 6.)

| | |
|---|---|
| SEQ ID NO: 1 | |
| 1 (ycdS) | |
| ATGTATTCAAGTAGCAGAAAAAGGTGCCCGAAAACCAAATGGGCTTTGAAACTTCTTACT | 300 |
| M Y S S S R K R C P K T K W A L K L L T | |
| GCCGCATTTTTAGCAGCGAGTCCCGCGGCGAAGAGTGCTGTTAATAACGCCTATGATGCA | 360 |
| A A F L A A S P A A K S A V N N A Y D A | |
| TTGATTATTGAAGCTCGCAAGGGTAATACTCAGCCAGCTTTGTCATGGTTTGCACTAAAA | 420 |
| L I I E A R K G N T Q P A L S W F A L K | |
| TCAGCACTCAGCAATAACCAAATTGCTGACTGGTTACAGATTGCCTTATGGGCCGGGCAA | 480 |
| S A L S N N Q I A D W L Q I A L W A G Q | |
| GATAAACAGGTTATTACCGTTTACAACCGCTACCGTCATCAGCAATTACCAGCGCGTGGT | 540 |
| D K Q V I T V Y N R Y R H Q Q L P A R G | |
| TATGCAGCTGTCGCCGTCGCTTATCGTAACCTGCAACAATGGCAAAACTCGCTTACACTG | 600 |
| Y A A V A V A Y R N L Q Q W Q N S L T L | |
| TGGCAAAAGGCGCTCTCTCTGGAGCCGCAAAATAAGGATTATCAACGGGGACAAATTTTA | 660 |
| W Q K A L S L E P Q N K D Y Q R G Q I L | |
| ACCCTGGCAGATGCTGGTCACTATGATACTGCGCTGGTTAAACTTAAGCAGCTTAACTCT | 720 |
| T L A D A G H Y D T A L V K L K Q L N S | |
| GGAGCACCGGACAAAGCCAATTTACTCGCAGAAGCCTATATCTATAAACTGGCGGGGCGT | 780 |
| G A P D K A N L L A E A Y I Y K L A G R | |
| CATCAGGATGAATTACGGGCGATGACAGAGTCATTACCTGAAAATGCATCTACGCAACAA | 840 |
| H Q D E L R A M T E S L P E N A S T Q Q | |
| TATCCCACAGAATACGTGCAGGCATTACGTAATAATCAACTTGCTGCCGCGATTGACGAT | 900 |
| Y P T E₇₅ Y V Q A L R N N Q L A A A I D D | |
| GCCAATTTAACGCCAGATATTCGCGCTGATATTCATGCCGAACTGGTCAGACTGTCGTTT | 960 |
| A N L T P D I R A D I H A E L V R L S F | |
| ATGCCTACGCGCAGTGAAAGTGAACGTTATGCCATTGCCGATCGCGCCCTCGCCCAATAC | 1020 |
| M P T R S E S E R Y A I A D R A L A Q Y | |
| GCTGCATTAGAAATTCTGTGGCACGATAACCCAGACCGCACTGCCCAGTACCAGCGTATT | 1080 |
| A A L E I L W H D N P D R T A Q Y Q R I | |
| CAGGTTGATCATCTTGGCGCGTTATTAACTCGCGATCGTTATAAAGACGTTATTTCTCAC | 1140 |
| Q V D H L G A L L T R D R Y K D V I S H | |
| TATCAGCGATTAAAAAAGACGGGGCAAATTATTCCGCCCTGGGGGCAATATTGGGTTGCA | 1200 |
| Y Q R L K K T G Q I I P P W G Q Y W V A | |
| TCGGCTTATCTCAAAGATCATCAGCCGAAAAAAGCACAGTCAATAATGACCGAGCTCTTT | 1260 |
| S A Y L K D H Q P K K A Q S I M T E L F | |
| TATCACAAGGAGACCATTGCCCCGGATTTATCCGATGAAGAACTTGCGGATCTCTTTTAC | 1320 |
| Y H K E T I A P D L S D E E L A D L F Y | |
| AGCCACCTGGAGAGTGAAAATTATCCGGGCGCGCTAACTGTCACCCAACATACCATTAAT | 1380 |
| S H L E S E N Y P G A L T V T Q H T I N | |
| ACTTCGCCGCCTTTCCTTCGGTTAATGGGCACGCCTACGAGCATCCCGAATGATACCTGG | 1440 |
| T S P P F L R L M G T P T S I P N D T W | |
| TTACAGGGGCATTCGTTTCTCTCAACCGTAGCAAAATATAGTAATGATCTTCCTCAGGCT | 1500 |
| L Q G H S F L S T V A K Y S N D L P Q A | |
| GAAATGACAGCCAGAGAGCTTGCTTATAACGCACCAGGAAATCAGGGACTGCGCATTGAT | 1560 |
| E M T A R E L A Y N A P G N Q G L R I D | |
| TACGCGAGTGTGTTACAAGCCCGCGGTTGGCCTCGTGCAGCAGAAAATGAATTAAAAAAA | 1620 |
| Y A S V L Q A R G W P R A A E N E L K K | |
| GCAGAAGTGATCGAGCCACGTAATATTAATCTGGAGGTTGAACAAGCCTGGACAGCATTA | 1680 |
| A E V I E P R N I N L E V E Q A W T A L | |
| ACGTTACAAGAATGGCAGCAGGCAGCTGTCTTAACGCACGATGTTGTCGAACGTGAACCG | 1740 |
| T L Q E W Q Q A A V L T H D V V E R E P | |
| CAAGATCCCGGCGTTGTACGATTAAAACGTGCGGTTGATGTACATAATCTTGCAGAGCTT | 1800 |
| Q D P G V V R L K R A V D V H N L A E L | |
| CGTATCGCTGGCTCAACAGGAATTGATGCCGAAGGCCCGGATAGTGGTAAACATGATGTC | 1860 |
| R I A G S T G I D A E G P D S G K H D V | |
| GACTTAACCACCATCGTTTATTCACCACCGCTGAAGGATAACTGGCGCGGTTTTGCTGGA | 1920 |
| D L T T I V Y S P P L K D N W R G F A G | |
| TTCGGTTATGCCGATGGACAATTTAGCGAAGGAAAAGGGATTGTTCGCGACTGGCTTGCG | 1980 |
| F G Y A D G Q F S E G K G I V R D W L A | |
| GGTGTTGAGTGGCGGTCACGTAATATCTGGCTCGAGGCAGAGTACGCTGAACGCGTTTTC | 2040 |
| G V E W R S R N I W L E A E Y A E R V F | |
| AATCATGAGCATAAACCCGGCGCGCGCCTGTCTGGCTGGTATGATTTTAATGATAACTGG | 2100 |
| N H E H K P G A R L S G W Y D F N D N W | |
| CGTATTGGTTCGCAACTGGAACGCCTCTCTCACCGCGTTCCATTACGGGCAATGAAAAAT | 2160 |
| R I G S Q L E R L S H R V P L R A M K N | |
| GGTGTTACAGGCAACAGTGCTCAGGCTTATGTTCGCTGGTATCAAAATGAGCGGCGTAAG | 2220 |
| G V T G N S A Q A Y V R W Y Q N E R R K | |
| TACGGTGTCTCCTGGGCTTTCACTGATTTTTCCGACAGTAACCAGCGTCATGAAGTCTCA | 2280 |
| Y G V S W A F T D F S D S N Q R H E V S | |
| CTTGAGGGTCAGGAACGCATCTGGTCTTCACCATATTTGATTGTCGATTTCCTACCCAGT | 2340 |
| L E G Q E R I W S S P Y L I V D F L P S | |
| CTGTATTACGAACAAAATACAGAACACGATACCCCATACTACAACCCTATAAAAACGTTC | 2400 |
| L Y Y E Q N T E H D T P Y Y N P I K T F | |
| GATATTGTTCCGGCATTTGAGGCAAGCCATTTGTTATGGCGAAGCTATGAAAATAGCTGG | 2460 |
| D I V P A F E A S H L L W R S Y E N S W | |
| GAGCAAATATTCAGCGCAGGTGTTGGTGCCTCCTGGCAAAAACATTATGGCACGGATGTC | 2520 |
| E Q I F S A G V G A S W Q K H Y G T D V | |
| GTCACCCAACTCGGCTACGGGCAACGCATTAGTTGGAATGACGTGATTGATGCTGGCGCA | 2580 |
| V T Q L G Y G Q R I S W N D V I D A G A | |
| ACGCTACGCTGGGAAAAACGACCTTATGACGGTGACAGAGAACACAACTTATACGTTGAA | 2640 |
| T L R W E K R P Y D G D R E H N L Y V E | |
| TTCGATATGACATTCAGATTTTAAGGATAAATATGTTACGTAATGGAAATAAATATCTCC | 2700 |
| F D M T F R F * | |
| SEQ ID NO: 2 | |
| *** | |
| 1(YCDR) | |
| TTAAGGATAAATATGTTACGTAATGGAAATAAATATCTCCTGATGCTGGTGAGTATAATT | 60 |
| M L R N G N K Y L L ML V S I I | |
| ATGCTCACCGCGTGCATTAGCCAGTCAAGAACATCATTTATACCGCCACAGGATCGCGAA | 120 |
| M L T A C I S Q S R T S F I P P Q D R E | |
| TCTTTACTCGCCGAGCAACCGTGGCCGCATAATGGTTTTGTAGCGATTTCATGGCATAAC | 180 |
| S L L A E Q P W P H N G F V A I S W H N | |
| GTTGAAGACGAAGCTGCCGACCAGCGTTTTATGTCAGTGCGGACATCAGCACTGCGTGAA | 240 |
| V E D E A A D Q R F M S V R T S A L R E | |
| CAATTTGCCTGGCTGCGCGAGAACGGTTATCAACCGGTCAGTATTGCTCAAATTCGTGAA | 300 |
| Q F A W L R E N G Y Q P V S I A Q I R E | |
| GCACATCGAGGAGGAAAACCGCTACCGGAAAAAGCTGTAGTGCTGACTTTTGATGACGGC | 360 |
| A H R G G K P L P E K A V V L T F D D G | |
| TACCAGAGTTTTTATACCCGCGTCTTCCCAATTCTTCAGGCCTTCCAGTGGCCTGCTGTA | 420 |
| Y Q S F Y T R V F P I L Q A F Q W P A V | |
| TGGGCCCCCGTCGGCAGTTGGGTCGATACGCCAGCGGATAAACAAGTAAAATTTGGCGAT | 480 |
| W A P V G S W V D T P A D K Q V K F G D | |
| GAGTTGGTCGATCGAGAATATTTTGCCACGTGGCAACAAGTGCGAGAAGTTGCGCGTTCC | 540 |
| E L V D R E Y F A T W Q Q V R E V A R S | |
| CGGCTCGTTGAGCTCGCTTCTCATACATGGAATTCTCACTACGGTATTCAGGCTAATGCC | 600 |
| R L V E L A S H T W N S H Y G I Q A N A | |
| ACCGGCAGCTTATTGCCTGTATATGTAAATCGTGCATATTTTACTGACCACGCACGGTAT | 660 |
| T G S L L P V Y V N R A Y F T D H A R Y | |
| GAAACCGCAGCAGAATACCGGGAAAGAATTCGTCTGGATGCTGTAAAAATGACGGAATAC | 720 |
| E T A A E Y R E R I R L D A V K M T E Y | |
| CTGCGTACAAAGGTTGAGGTAAATCCACACGTTTTTGTTTGGCCTTATGGCGAAGCGAAT | 780 |
| L R T K V E V N P H V F V W P Y G E A N | |
| GGCATAGCGATAGAGGAATTAAAAAAACTCGGTTATGACATGTTCTTCACCCTTGAATCA | 840 |
| G I A I E E L K K L G Y D M F F T L E S | |
| GGTTTGGCAAATGCGTCGCAATTGGATTCCATTCCGCGGGTATTAATCGCCAATAATCCC | 900 |
| G L A N A S Q L D S I P R V L I A N N P | |
| TCATTAAAAGAGTTTGCCCAGCAAATTATTACCGTACAGGAAAAATCACCACAACGGATA | 960 |
| S L K E F A Q Q I I T V Q E K S P Q R I | |
| ATGCATATCGATCTTGATTACGTTTATGACGAAAACCTCCAGCAAATGGATCGCAATATT | 1020 |
| M H I D L D Y V Y D E N L Q Q M D R N I | |
| GATGTGCTAATTCAGCGGGTGAAAGATATGCAAATATCAACCGTGTATTTGCAGGCATTT | 1080 |
| D V L I Q R V K D M Q I S T V Y L Q A F | |
| GCTGATCCCGATGGTGATGGGCTGGTCAAAGAGGTCTGGTTTCCAAATCGTTTGCTACCA | 1140 |
| A D P D G D G L V K E V W F P N R L L P | |
| ATGAAAGCAGATATTTTTAGTCGGGTTGCCTGGCAATTACGTACCCGCTCAGGTGTAAAC | 1200 |
| M K A D I F S R V A W Q L R T R S G V N | |
| ATCTATGCGTGGATGCCGGTATTAAGCTGGGATTTAGATCCCACATTAACGCGAGTAAAA | 1260 |
| I Y A W M P V L S W D L D P T L T R V K | |
| TACTTACCAACAGGGGAGAAAAAAGCACAAATTCATCCTGAACAATATCACCGTCTCTCT | 1320 |
| Y L P T G E K K A Q I H P E Q Y H R L S | |
| CCTTTCGATGACAGAGTCAGAGCACAAGTTGGCATGTTATATGAAGATCTTGCCGGACAT | 1380 |
| P F D D R V R A Q V G M L Y E D L A G H | |
| GCTGCTTTTGATGGCATATTGTTCCACGATGATGCTTTGCTTTCAGATTATGAAGATGCC | 1440 |
| A A F D G I L F H D D A L L S D Y E D A | |
| AGTGCACCGGCTATCACGGCTTATCAGCAAGCAGGCTTTAGCGGGAGTCTGAGCGAAATT | 1500 |
| S A P A I T A Y Q Q A G F S G S L S E I | |
| CGACAAAACCCGGAGCAATTTAAACAGTGGGCCCGCTTTAAAAGTCGTGCGTTAACTGAC | 1560 |
| R Q N P E Q F K Q W A R F K S R A L T D | |
| TTCACTTTAGAACTTAGTGCGCGCGTAAAAGCCATTCGCGGTCCACATATTAAAACTGCA | 1620 |
| F T L E L S A R V K A I R G P H I K T A | |
| CGAAATATTTTTGCACTTCCGGTAATACAACCTGAAAGTGAAGCCTGGTTTGCACAGAAT | 1680 |
| R N I F A L P V I Q P E S E A W F A Q N | |
| TATGCTGATTTCCTAAAAAGCTATGACTGGACCGCTATTATGGCTATGCCTTATCTGGAA | 1740 |
| Y A D F L K S Y D W T A I M A M P Y L E | |
| GGTGTCGCAGAAAAATCGGCTGACCAATGGTTAATACAATTGACCAATCAAATTAAAAAC | 1800 |
| G V A E K S A D Q W L I Q L T N Q I K N | |
| ATCCCTCAGGCTAAAGACAAATCTATTTTAGAATTACAGGCACAAAACTGGCAGAAAAAT | 1860 |
| I P Q A K D K S I L E L Q A Q N W Q K N | |
| GGTCAGCATCAGGCTATTTCTTCGCAACAACTCGCTCACTGGATGAGCCTATTACAACTG | 1920 |
| G Q H Q A I S S Q Q L A H W M S L L Q L | |
| AATGGAGTGAAAAACTATGGTTATTATCCCGACAATTTTCTGCATAACCAACCTGAAATA | 1980 |
| N G V K N Y G Y Y P D N F L H N Q P E I | |
| GACCTTATTCGTCCTGAGTTTTCAACAGCCTGGTATCCGAAAAATGATTAA | 2031 |
| D L I R P E F S T A W Y P K N D *** | |
| (YCDR STOP CODON) | |
| (YCDQ START CODON) | |
| SEQ ID NO: 3 | |
| 1 (ycdQ) * | |
| AAAATGATTAATCGCATCGTATCGTTTTTTATATTATGTCTGGTGTTATGCATACCCCTA | 240 |
| M I N R I V S F F I L C L V L C I P L | |
| TGCGTAGCGTACTTTCACTCTGGTGAACTGATGATGAGGTTCGTTTTCTTCTGGCCGTTT | 300 |
| C V A Y F H S G E L M M R F V F F W P F | |
| TTTATGTCCATTATGTGGATTGTTGGCGGCGTCTATTTCTGGGTCTATCGTGAACGCCAC | 360 |
| F M S I M W I V G G V Y F W V Y R E R H | |
| TGGCCGTGGGGAGAAAACGCACCAGCTCCCCAGTTGAAAGATAATCCGTCTATCTCCATT | 420 |
| W P W G E N A P A P Q L K D N P S I S I | |
| ATCATTCCCTGTTTTAATGAGGAGAAAAACGTTGAGGAAACCATACACGCCGCTTTAGCA | 480 |
| I I P C F N E E K N V E E T I H A A L A | |
| CAGCGTTATGAGAACATTGAAGTTATTGCCGTAAATGACGGTTCAACAGATAAAACCCGT 540 | |
| Q R Y E N I E V I A V N D G S T D K T R | |
| GCCATCCTGGATCGCATGGCTGCACAAATTCCCCATTTGCGGGTCATTCATCTGGCGCAA | 600 |
| A I L D R M A A Q I P H L R V I H L A Q | |
| AACCAGGGGAAAGCCATTGCGCTTAAAACCGGAGCTGCCGCGGCGAAAAGTGAATATCTG | 660 |
| N Q G K A I A L K T G A A A A K S E Y L | |
| GTGTGCATTGATGGCGATGCGTTATTAGACCGCGATGCGGCGGCATATATTGTGGAACCG | 720 |
| V C I D G D A L L D R D A A A Y I V E P | |
| ATGTTGTACAACCCGCGTGTGGGTGCCGTAACCGGTAATCCTCGTATTCGAACACGTTCT | 780 |
| M L Y N P R V G A V T G N P R I R T R S | |
| ACCCTGGTGGGTAAAATTCAGGTTGGCGAGTATTCCTCAATTATTGGTTTGATCAAGCGA | 840 |
| T L V G K I Q V G E Y S S I I G L I K R | |
| ACCCAGCGTATCTATGGAAACGTATTTACCGTTTCCGGTGTTATTGCCGCATTTCGTCGC | 900 |
| T Q R I Y G N V F T V S G V I A A F R R | |
| AGCGCCCTGGCAGAAGTGGGTTACTGGAGTGACGATATGATCACCGAAGATATTGATATT | 960 |
| S A L A E V G Y W S D D M I T E D I D I | |
| AGCTGGAAGCTGCAGTTGAATCAGTGGACGATTTTTTACGAGCCACGGGCACTGTGCTGG | 1020 |
| S W K L Q L N Q W T I F Y E_{↓} P R A L C W | |
| ATATTAATGCCTGAAACGTTAAAAGGGCTGTGGAAACAGCGCCTGCGCTGGGCTCAGGGC | 1080 |
| I L M P E T L K G L W K Q R L R W A Q G | |
| GGTGCAGAAGTATTCCTCAAAAATATGACAAGGTTGTGGCGCAAAGAAAACTTTCGAATG | 1140 |
| G A E V F L K N M T R L W R K E N F R M | |
| TGGCCGCTGTTTTTTGAATACTGCCTGACGACAATATGGGCCTTCACCTGCCTGGTCGGT | 1200 |
| W P L F F E Y C L T T I W A F T C L V G | |
| TTCATTATTTACGCAGTCCAACTTGCCGGTGTACCGTTAAATATTGAATTGACACATATC | 1260 |
| F I I Y A V Q L A G V P L N I E L T H I | |
| GCTGCGACACATACTGCCGGAATATTATTGTGTACGTTATGTTTACTGCAATTTATTGTC | 1320 |
| A A T H T A G I L L C T L C L L Q F I V | |
| AGCCTGATGATCGAGAATCGCTATGAGCATAATCTGACTTCATCGCTTTTCTGGATTATT | 1380 |
| S L M I E N R Y E H N L T S S L F W I I | |
| TGGTTCCCGGTTATTTTCTGGATGCTGAGCCTGGCAACGACATTGGTATCATTTACACGA | 1440 |
| W F P V I F W M L S L A T T L V S F T R | |
| GTCATGTTGATGCCTAAAAAGCAACGCGCCCGTTGGGTAAGTCCCGATCGCGGGATTCTG | 1500 |
| V M L M P K K Q R A R W V S P D R G I L | |
| AGAGGTTAATATGAACAATTTAATTATTACGACCCGACAATCACCAGTACGTTTACTGGT | 1560 |
| R G * M N N L(ycdp) | |
| SEQ ID NO: 6 | |
| ycdS(+1) | |
| ATGTATTCAAGTAGCAGAAAAAGGTGCCCGAAAACCAAATGGGCTTTGAAACTTCTTACT | |
| GCCGCATTTTTAGCAGCGAGTCCCGCGGCGAAGAGTGCTGTTAATAACGCCTATGATGCA | |
| TTGATTATTGAAGCTCGCAAGGGTAATACTCAGCCAGCTTTGTCATGGTTTGCACTAAAA | |
| TCAGCACTCAGCAATAACCAAATTGCTGACTGGTTACAGATTGCCTTATGGGCCGGGCAA | |
| GATAAACAGGTTATTACCGTTTACAACCGCTACCGTCATCAGCAATTACCAGCGCGTGGT | 300 |
| TATGCAGCTGTCGCCGTCGCTTATCGTAACCTGCAACAATGGCAAAACTCGCTTACACTG | |
| 389 | |
| TGGCAAAAGGCGCTCTCTCTGGAGCCGC [symbol] AAATAAGGATTATCAACGGGGACAAATTTTA | |
| ACCCTGGCAGATGCTGGTCACTATGATACTGCGCTGGTTAAACTTAAGCAGCTTAACTCT | |
| GGAGCACCGGACAAAGCCAATTTACTCGCAGAAGCCTATATCTATAAACTGGCGGGGCGT | |
| 583 | |
| CATCAGGATGAATTACGGGCGATGACAGAGTCATTACCTGAAz,801 ATGCATCTACGCAACAA | 600 |
| TATCCCACAGAATACGTGCAGGCATTACGTAATAATCAACTTGCTGCCGCGATTGACGAT | |
| ↓ | |
| GCCAATTTAACGCCAGATATTCGCGCTGATATTCATGCCGAACTGGTCAGACTGTCGTTT | |
| ATGCCTACGCGCAGTGAAAGTGAACGTTATGCCATTGCCGATCGCGCCCTCGCCCAATAC | |
| GCTGCATTAGAAATTCTGTGGCACGATAACCCAGACCGCACTGCCCAGTACCAGCGTATT | |
| CAGGTTGATCATCTTGGCGCGTTATTAACTCGCGATCGTTATAAAGACGTTATTTCTCAC | 900 |
| TATCAGCGATTAAAAAAGACGGGGCAAATTATTCCGCCCTGGGGGCAATATTGGGTTGCA | |
| TCGGCTTATCTCAAAGATCATCAGCCGAAAAAAGCACAGTCAATAATGACCGAGCTCTTT | |
| TATCACAAGGAGACCATTGCCCCGGATTTTATCCGATGAAGAACTTGCGGATCTCTTTTAC | |
| AGCCACCTGGAGAGTGAAAATTATCCGGGCGCGCTAACTGTCACCCAACATACCATTAAT | |
| ACTTCGCCGCCTTTCCTTCGGTTAATGGGCACGCCTACGAGCATCCCGAATGATACCTGG | 1200 |
| TTACAGGGGCATTCGTTTCTCTCAACCGTAGCAAAATATAGTAATGATCTTCCTCAGGCT | |
| GAAATGACAGCCAGAGAGCTTGCTTATAACGCACCAGGAAATCAGGGACTGCGCATTGAT | |
| TACGCGAGTGTGTTACAAGCCCGCGGTTGGCCTCGTGCAGCAGAAAATGAATTAAAAAAA | |
| GCAGAAGTGATCGAGCCACGTAATATTAATCTGGAGGTTGAACAAGCCTGGACAGCATTA | |
| ACGTTACAAGAATGGCAGCAGGCAGCTGTCTTAACGCACGATGTTGTCGAACGTGAACCG | 1500 |
| CAAGATCCCGGCGTTGTACGATTAAAACGTGCGGTTGATGTACATAATCTTGCAGAGCTT | |
| CGTATCGCTGGCTCAACAGGAATTGATGCCGAAGGCCCGGATAGTGGTAAACATGATGTC | |
| GACTTAACCACCATCGTTTATTCACCACCGCTGAAGGATAACTGGCGCGGTTTTGCTGGA | |
| TTCGGTTATGCCGATGGACAATTTAGCGAAGGAAAAGGGATTGTTCGCGACTGGCTTGCG | |
| GGTGTTGAGTGGCGGTCACGTAATATCTGGCTCGAGGCAGAGTACGCTGAACGCGTTTTC | 1800 |
| AATCATGAGCATAAACCCGGCGCGCGCCTGTCTGGCTGGTATGATTTTAATGATAACTGG | |
| CGTATTGGTTCGCAACTGGAACGCCTCTCTCACCGCGTTCCATTACGGGCAATGAAAAAT | |
| GGTGTTACAGGCAACAGTGCTCAGGCTTATGTTCGCTGGTATCAAAATGAGCGGCGTAAG | |
| TACGGTGTCTCCTGGGCTTTCACTGATTTTTCCGACAGTAACCAGCGTCATGAAGTCTCA | |
| CTTGAGGGTCAGGAACGCATCTGGTCTTCACCATATTTGATTGTCGATTTCCTACCCAGT | 2100 |
| CTGTATTACGAACAAAATACAGAACACGATACCCCATACTACAACCCTATAAAAACGTTC | |
| GATATTGTTCCGGCATTTGAGGCAAGCCATTTGTTATGGCGAAGCTATGAAAATAGCTGG | |
| GAGCAAATATTCAGCGCAGGTGTTGGTGCCTCCTGGCAAAAACATTATGGCACGGATGTC | |
| GTCACCCAACTCGGCTACGGGCAACGCATTAGTTGGAATGACGTGATTGATGCTGGCGCA | |
| ACGCTACGCTGGGAAAAACGACCTTATGACGGTGACAGAGAACACAACTTATACGTTGAA | 2400 |
| ycdR(+1) | |
| TTCGATATGACATTCAGATTTTAAGGATAAATATGTTACGTAATGGAAATAAATATCTCC | |
| TGATGCTGGTGAGTATAATTATGCTCACCGCGTGCATTAGCCAGTCAAGAACATCATTTA | |
| TACCGCCACAGGATCGCGAATCTTTACTCGCCGAGCAACCGTGGCCGCATAATGGTTTTG | |
| TAGCGATTTCATGGCATAACGTTGAAGACGAAGCTGCCGACCAGCGTTTTATGTCAGTGC | |
| GGACATCAGCACTGCGTGAACAATTTGCCTGGCTGCGCGAGAACGGTTATCAACCGGTCA | 2700 |
| GTATTGCTCAAATTCGTGAAGCACATCGAGGAGGAAAACCGCTACCGGAAAAAGCTGTAG | |
| TGCTGACTTTTGATGACGGCTACCAGAGTTTTTATACCCGCGTCTTCCCAATTCTTCAGG | |
| CCTTCCAGTGGCCTGCTGTATGGGCCCCCGTCGGCAGTTGGGTCGATACGCCAGCGGATA | |
| AACAAGTAAAATTTGGCGATGAGTTGGTCGATCGAGAATATTTTGCCACGTGGCAACAAG | |
| ↓ | |
| TGCGAGAAGTTGCGCGTTCCCGGCTCGTTGAGCTCGCTTCTCATACATGGAATTCTCACT | 3000 |
| ACGGTATTCAGGCTAATGCCACCGGCAGCTTATTGCCTGTATATGTAAATCGTGCATATT | |
| TTACTGACCACGCACGGTATGAAACCGCAGCAGAATACCGGGAAAGAATTCGTCTGGATG | |
| 723 | |
| CTGTAAAAATGACGGAATACCTGCGTACAAAGGT [symbol] GAGGTAAATCCACACGTTTTTGTTT | |
| GGCCTTATGGCGAAGCGAATGGCATAGCGATAGAGGAATTAAAAAAACTCGGTTATGACA | |
| TGTTCTTCACCCTTGAATCAGGTTTGGCAAATGCGTCGCAATTGGATTCCATTCCGCGGG | 3300 |
| TATTAATCGCCAATAATCCCTCATTAAAAGAGTTTGCCCAGCAAATTATTACCGTACAGG | |
| AAAAATCACCACAACGGATAATGCATATCGATCTTGATTACGTTTATGACGAAAACCTCC | |
| AGCAAATGGATCGCAATATTGATGTGCTAATTCAGCGGGTGAAAGATATGCAAATATCAA | |
| CCGTGTATTTGCAGGCATTTGCTGATCCCGATGGTGATGGGCTGGTCAAAGAGGTCTGGT | |
| TTCCAAATCGTTTGCTACCAATGAAAGCAGATATTTTTAGTCGGGTTGCCTGGCAATTAC | 3600 |
| GTACCCGCTCAGGTGTAAACATCTATGCGTGGATGCCGGTATTAAGCTGGGATTTAGATC | |
| CCACATTAACGCGAGTAAAATACTTACCAACAGGGGAGAAAAAAGCACAAATTCATCCTG | |
| AACAATATCACCGTCTCTCTCCTTTCGATGACAGAGTCAGAGCACAAGTTGGCATGTTAT | |
| ATGAAGATCTTGCCGGACATGCTGCTTTTGATGGCATATTGTTCCACGATGATGCTTTGC | |
| TTTCAGATTATGAAGATGCCAGTGCACCGGCTATCACGGCTTATCAGCAAGCAGGCTTTA | 3900 |
| GCGGGAGTCTGAGCGAAATTCGACAAAACCCGGAGCAATTTAAACAGTGGGCCCGCTTTA | |
| AAAGTCGTGCGTTAACTGACTTCACTTTAGAACTTAGTGCGCGCGTAAAAGCCATTCGCG | |
| GTCCACATATTAAAACTGCACGAAATATTTTTGCACTTCCGGTAATACAACCTGAAAGTG | |
| AAGCCTGGTTTGCACAGAATTATGCTGATTTCCTAAAAAGCTATGACTGGACCGCTATTA | |
| TGGCTATGCCTTATCTGGAAGGTGTCGCAGAAAAATCGGCTGACCAATGGTTAATACAAT | 4200 |
| TGACCAATCAAATTAAAAACATCCCTCAGGCTAAAGACAAATCTATTTTAGAATTACAGG | |
| CACAAAACTGGCAGAAAAATGGTCAGCATCAGGCTATTTCTTCGCAACAACTCGCTCACT | |
| GGATGAGCCTATTACAACTGAATGGAGTGAAAAACTATGGTTATTATCCCGACAATTTTC | |
| TGCATAACCAACCTGAAATAGACCTTATTCGTCCTGAGTTTTCAACAGCCTGGTATCCGA | |
| ycdQ(+1) | |
| AAAATGATTAATCGCATCGTATCGTTTTTTATATTATGTCTGGTGTTATGCATACCCCTA | 4500 |
| TGCGTAGCGTACTTTCACTCTGGTGAACTGATGATGAGGTTCGTTTTCTTCTGGCCGTTT | |
| TTTATGTCCATTATGTGGATTGTTGGCGGCGTCTATTTCTGGGTCTATCGTGAACGCCAC | |
| TGGCCGTGGGGAGAAAACGCACCAGCTCCCCAGTTGAAAGATAATCCGTCTATCTCCATT | |
| ATCATTCCCTGTTTTAATGAGGAGAAAAACGTTGAGGAAACCATACACGCCGCTTTAGCA | |
| CAGCGTTATGAGAACATTGAAGTTATTGCCGTAAATGACGGTTCAACAGATAAAACCCGT | 4800 |
| GCCATCCTGGATCGCATGGCTGCACAAATTCCCCATTTGCGGGTCATTCATCTGGCGCAA | |
| AACCAGGGGAAAGCCATTGCGCTTAAAACCGGAGCTGCCGCGGCGAAAAGTGAATATCTG | |
| GTGTGCATTGATGGCGATGCGTTATTAGACCGCGATGCGGCGGCATATATTGTGGAACCG | |
| ATGTTGTACAACCCGCGTGTGGGTGCCGTAACCGGTAATCCTCGTATTCGAACACGTTCT | |
| ACCCTGGTGGGTAAAATTCAGGTTGGCGAGTATTCCTCAATTATTGGTTTGATCAAGCGA | 5100 |
| ACCCAGCGTATCTATGGAAACGTATTTACCGTTTCCGGTGTTATTGCCGCATTTCGTCGC | |
| AGCGCCCTGGCAGAAGTGGGTTACTGGAGTGACGATATGATCACCGAAGATATTGATATT | |
| AGCTGGAAGCTGCAGTTGAATCAGTGGACGATTTTTTACGAGCCACGGGCACTGTGCTGG | |
| ↓ | |
| ATATTAATGCCTGAAACGTTAAAAGGGCTGTGGAAACAGCGCCTGCGCTGGGCTCAGGGC | |
| GGTGCAGAAGTATTCCTCAAAAATATGACAAGGTTGTGGCGCAAAGAAAACTTTCGAATG | 5400 |
| TGGCCGCTGTTTTTTGAATACTGCCTGACGACAATATGGGCCTTCACCTGCCTGGTCGGT | |
| TTCATTATTTACGCAGTCCAACTTGCCGGTGTACCGTTAAATATTGAATTGACACATATC | |
| GCTGCGACACATACTGCCGGAATATTATTGTGTACGTTATGTTTACTGCAATTTATTGTC | |
| AGCCTGATGATCGAGAATCGCTATGAGCATAATCTGACTTCATCGCTTTTCTGGATTATT | |
| TGGTTCCCGGTTATTTTCTGGATGCTGAGCCTGGCAACGACATTGGTATCATTTACACGA | 5700 |
| GTCATGTTGATGCCTAAAAAGCAACGCGCCCGTTGGGTAAGTCCCGATCGCGGGATTCTG | |
| ycdP(+1) | |
| AGAGGTTAATATGAACAATTTAATTATTACGACCCGACAATCACCAGTACGTTTACTGGT | |
| TGATTATGTTGCCACAACCATCTTGTGGACATTATTTGCGTTGTTCATATTCTTATTCGC | |
| ↓ | |
| CATGGATCTGCTGACGGGTTATTACTGGCAAAGCGAGGCCAGAAGCCGACTTCAGTTCTA | |
| TTTTTTGCTGGCAGTGGCGAATGCCGTCGTGTTAATTGTCTGGGCGCTGTACAATAAGCT | 6000 |
| GCGTTTTCAAAAACAGCAGCATCATGCAGCCTACCAATATACGCCGCAAGAATATGCAGA | |
| GAGCTTAGCAATACCTGATGAGCTCTATCAGCAACTACAAAAAAGCCACAGGATGAGCGT | |
| ACACTTCACCAGCCAGGGGCAAATAAAAATGGTTGTTTCAGAAAAAGCGCTAGTCCGGGC | |
| ATAAACACCCAAAACAAAGCCCGGTTCGCCCGGGCTCTGCACCGATAACACACTTAACTG | |
| TAGGCATGCAGCGTACGTTGGCAAAGTGCCGAACGTACGCAGTCCTCTTTACCGAACCGG | 6300 |
| ACGATCCCAACCATTTCATCTTCTTCGAAACGTTCCAGCGCGTCACTTAATCCGGAGCAC | |
| ACGCCGCGAGGCAAATCGCATTGCGTGATATCACCGTTGACGATAACCGTCACGTTCTCC | |
| CCGAGGCGGGTTAAAAACATTTTCATTTGCGCGGCAGTCACATTCTGCGCCTCGTCAAGA | |
| ATGACGACTGCATTTTCAAAGGTACGTCCACGCATATAGGCGAACGGCGCAATTTCCACC | |
| TTCCCTATTTCCGGTCGCAGGCAGTACTGCATAAAGGAAGCCCCTAAGCGCCGGACCAGC | 6600 |
| ACGTCGTAGACCGGGCGAAAATAGGGAGCAAACTTTTCTGCGATATCTCCAGGTAAGAAG | |
| CCAAGATCTTCATCGGCTTGCAGAACTGGACGGGTGACGATAATCCTGTCGACATCCTTA | |
| TGTATCAGGGCCTCTGCCGCTTTTGCTGCGCTGATCCAGGTTTTTCCGCACCCGGCTTCG | |
| CCCGTGGCGAATATCAGCTGCTTACTCTCAATAGCCTTCAGATAGTGCAATTGCGCTTCA | |
| TTTCGCGCGAGGATGGGCGAAGTATCGCGACTGTCGCGGGCCATACCAATGGCTTCTACG | 6900 |
| CCGCCCATCTGCACAAGCGAGGTGACCGATTCTTCTTCACGCTGCTTATGGCTGCGCGAA | |
| TCCCGTCTCAGCACACGTTTTGCCTCGCGACGAGCTTTGATCACTGCTTTTTGTCTTCCC | |
| ATGGAGAGCACCTTGAGTTGTTTGTATTCATCACACGCGCCGTTGGCAGCGCGATTATGC | |
| GCACGAACATCAGAGGGTTGGCTTCCTTGTAAGCCATAGTTTGCTTTTGGATAAAATGCC | |
| GAAAAACGGCTACGCGCACCGTTTACGGCGTCGGTAACACATGAAAAGAAAGGATGAGGT | 7200 |
| TGAAAATGCAAAGTGACGAGATGACTACCGGAGGAGAAAACTCCGCGAGTGGTGGCGCGT | |
| TGATTATCTAAAACATGTCCAGTACAGGACGTTACCATCCGCGATCTCCATAGTGACTGA | |
| CTATCACTGCCGGGAACTTCCGCTGCTACTTAATAAGTACAACAGATCTCGCATTTATTG | |
| CAACAATATATTTACTTATATTTAACTATAAAACACCATTTCAGTGACATTAGTTTCTAC | |
| TGGAAAGATGACAGAGTGATGACAGTGATGAAAAAAGCTGTGTGCTTTCAGCAGGATTTG | 7500 |

Note:
Larger letters indicate mutated nucleotides in cloned ycd operon carried by pUCPG372.
Arrows indicate the locations of insertion.

Mutations in cloned ycd operon carried by pUCPGA372. One mutation is in the ycdR gene, in which nucleotide 723 was changed from T to C, and the codon was changed from TTG (Leu) to TCG (Ser). The other two mutations are in ycdS gene, in which nucleotide 583 and 389 were changed from A to G, and the codons were changed from AAT (Asn) to GAT (Asp), and CAA (Gin) to CGA (Arg) respectively.

We claim:

1. Use of an isolated polynucleotide sequence encoding at least 200 amino acids having a sequence found in SEQ ID NO: 1 in the preparation of a medicament useful in the modulation of polysaccharide adhesin synthesis.

2. Use of claim 1 wherein the polynucleotide sequence is a DNA sequence.

3. Use of claim 1 wherein the polynucleotide sequence is a RNA sequence.

4. Use of an isolated polynucleotide sequence encoding at least 200 amino acids having a sequence found in SEQ ID NO: 2 in the preparation of a medicament useful in the modulation of polysaccharide adhesin synthesis.

5. Use of claim 4 wherein the polynucleotide sequence is a DNA sequence.

6. Use of claim 4 wherein the polynucleotide sequence is a RNA sequence.

7. Use of an isolated polynucleotide sequence encoding at least 200 amino acids having a sequence found in SEQ ID NO: 3 in the preparation of a medicament useful in the modulation of polysaccharide adhesin synthesis.

8. Use of claim 7 wherein the polynucleotide sequence is a DNA sequence.

9. Use of claim 7 wherein the polynucleotide sequence is a RNA sequence.

10. Use of an isolated amino acid sequence comprising at least 200 amino acids having a sequence found in at least one of SEQ ID NOs: 1, 2 or 3 in modulating polysaccharide adhesin synthesis by biofilm-producing bacteria.

11. Use of claim 10 wherein the sequence is a sequence found in SEQ ID NO: 1.

12. Use of claim 10 wherein the sequence is a sequence found in SEQ ID NO: 2.

13. Use of claim 10 wherein the sequence is a sequence found in SEQ ID NO: 3.

14. A method of identifying inhibitors of a product of the ycdSRQP operon, comprising selecting the product, assaying the activity of that product under controlled conditions, adding a potential inhibitor of the product, assaying the activity of the product in the presence of the potential inhibitor, and ascertaining whether the presence of the proposed inhibitor resulted in a n inhibition of the function of that product.

15. The method of claim 14 wherein the product of the ycdSRQP operon is ycdQ.

16. The method of claim 14 wherein the product of the ycdSRQP operon is ycdR.

17. The method of claim 14 wherein the product of the ycdSRQP operon is ycdS.

18. A method of reducing the rate of conversion of UDP-GlcNAc to β-1,6-GlcNAa polymeric units in an environment containing biofilm-producing bacteria, comprising reducing the expression of a product of the ycdSRQP operon.

19. The method of claim 18 wherein the product of the ycdSRQP operon is YcdQ.

20. The method of claim 18 wherein the product of the ycdSRQP operon is YcdR.

21. The method of claim 18 wherein the product of the ycdSRQP operon is YcdR.

22. A method of inhibiting polysaccharide deacetylation by reducing YcdR activity.

23. The method of claim 22 wherein YcdR activity is reduced in E. coli.

24. A method of inhibiting adhesin transport in biofilm-producing bacteria comprising reducing YcdR activity.

25. The method of claim 24 wherein the biofilm-producing bacteria is E. coli.

26. A method of reducing extracellular adhesin binding in biofilm-producing bacteria, comprising reducing YcdS activity.

27. Use of an inhibitor of a product of the ycdSRQP operon in improving the response of a mammalian patient suffering from a bacterial infection to antibiotics for treatment of said bacterial infection.

28. Use of claim 27 wherein the mammalian patient is a human.

29. Use of an inhibitor of the expression of a product of the ycdSRQP operon in facilitating the reduction of bacterial load in a mammalian patient suffering from bacterial infection by biofilm-forming bacteria.

30. Use of claim 29 wherein the mammalian patient is a human.

31. The method of claim 18 wherein the biofilm-producing bacteria includes E. coli.

32. A method of decreasing cell to cell biofilm links in biofilm-forming bacteria, comprising reducing YcdS activity.

33. A method of reducing adhesin synthesis in biofilm-forming bacteria, by reducing YcdQ activity.

34. A method of reducing β-1,6-N-acetylglucosamine polymer synthesis by reducing YcdQ activity.

35. A method of reducing glycosyltransferase activity in biofilm-forming bacteria, comprising reducing YcdQ activity.

36. The method of claim 32 wherein the biofilm-forming bacteria is at least one of E. coli or Staphylococcus.

37. An isolated polynucleotide sequence encoding at least 200 amino acids having a sequence found in SEQ ID NO: 1.

38. The polynucleotide sequence of claim 37 wherein the polynucleotide sequence is a DNA sequence.

39. The polynucleotide sequence of claim 37 wherein the polynucleotide sequence is a RNA sequence.

40. An isolated polynucleotide sequence encoding at least 200 amino acids having a sequence found in SEQ ID NO: 2.

41. The polynucleotide sequence of claim 40 wherein the polynucleotide sequence is a DNA sequence.

42. The polynucleotide sequence of claim 40 wherein the polynucleotide sequence is a RNA sequence.

43. An isolated polynucleotide sequence encoding at least 200 amino acids having a sequence found in SEQ ID NO: 3.

44. The polynucleotide sequence of claim 43 wherein the polynucleotide sequence is a DNA sequence.

45. The polynucleotide sequence of claim 43 wherein the polynucleotide sequence is a RNA sequence.
